(19) United States
(12) Patent Application Publication
(10) Pub. No.: US 2006/0084090 A1
(43) Pub. Date: Apr. 20, 2006

(54) Modulation of interaction between Nod1 and Gram-negative bacteria by Nod1 response to peptidoglycan delivered by the Helicobacter pylori cag pathogenicity island (cagPAI)

(76) Inventors: Richard Ferrero, Victoria (AU); Dana Philpott, Vincennes (FR); Jerome Viala, Ecouen (FR); Ivo Boneca, Vitry Sur Seine (FR); Catherine Chaput, Paris (FR); Stephen Girardin, Vincennes (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/195,696

(22) Filed: Aug. 3, 2005

Related U.S. Application Data
(63) Continuation of application No. 10/956,246, filed on Oct. 4, 2004, now abandoned.
Application No. 10/956,246 is a continuation-in-part of application No. 10/808,735, filed on Mar. 25, 2004.
(60) Provisional application No. 60/457,572, filed on Mar. 27, 2003.

(51) Int. Cl. C12Q 1/68; C12Q 1/18

(52) U.S. Cl. 435/6; 435/32

(57) ABSTRACT

A method for identifying a compound that modulates the interaction between Nod1 and a Gram-negative bacteria comprising contacting a Nod1 expressing cell with a cagPAI-positive H. pylori in the presence of a compound, contacting a Nod1 expressing cell with a cagPAI-positive H. pylori in the absence of said compound, and detecting the activation of a pro-inflammatory factor and/or the production of a pro-inflammatory cytokine or chemokine, wherein altered activation and/or production indicates that said compound modulates the interaction between Nod1 and the Gram-negative bacteria. The method is useful for identifying compounds for regulating the signaling cascade involving Nod1 against Gram-negative bacteria that cause mucosal inflammation in animal hosts.

### CROSS-REFERENCE TO RELATED APPLICATION

This application is a Continuation-In-Part of U.S. application Ser. No. 10/808,735, filed Mar. 25, 2004, Attorney Docket No. 03495.0308, which is based on and claims the benefit of U.S. Provisional Application Ser. No. 60/457,572, filed Mar. 27, 2003 (Attorney Docket No. 03495.6088). The entire disclosure of these applications are relied upon and incorporated by reference herein.

### BACKGROUND OF THE INVENTION

This invention relates to the first described pathogen-recognition molecule Nod1, which senses specifically Gram-negative bacteria through a peptidoglycan motif, muramyl tripeptide. More particularly, this invention relates to the modulation of Nod1 activity by a molecule related to the muramyl tripeptide (MTP). The invention also relates to a screening process for identifying a molecule capable of modulating Nod1 activity and the therapeutic use of such a molecule for modulating inflammation and/or apoptosis. The invention also relates to a new compound, which can be used for modulating inflammation and/or apoptosis or as an adjuvant agent.

In multicellular organisms, homeostasis is maintained by balancing the rate of cell proliferation against the rate of cell death. Cell proliferation is influenced by numerous growth factors and the expression of proto-oncogenes, which typically encourage progression through the cell cycle. In contrast, numerous events, including the expression of tumor suppressor genes, can lead to an arrest of cellular proliferation.

In differentiated cells, a particular type of cell death called apoptosis occurs when an internal suicide program is activated. This program can be initiated by a variety of external signals as well as signals that are generated within the cell in response to, for example, genetic damage. For many years, the magnitude of apoptotic cell death was not appreciated because the dying cells are quickly eliminated by phagocytes, without an inflammatory response.

The mechanisms that mediate apoptosis have been intensively studied. These mechanisms involve the activation of endogenous proteases, loss of mitochondrial function, and structural changes, such as disruption of the cytoskeleton, cell shrinkage, membrane blebbing, and nuclear condensation due to degradation of DNA. The various signals that trigger apoptosis are thought to bring about these events by converging on a common cell death pathway that is regulated by the expression of genes that are highly conserved from worms, such as C. elegans, to humans. In fact, invertebrate model systems have been invaluable tools in identifying and characterizing the genes that control apoptosis. Through the study of invertebrates and more evolved animals, numerous genes that are associated with cell death have been identified, but the way in which their products interact to execute the apoptotic program is poorly understood.

Caspases, a class of proteins central to the apoptotic program, are cysteine proteases having specificity for aspartate at the substrate cleavage site. These proteases are primarily responsible for the degradation of cellular proteins that lead to the morphological changes seen in cells undergoing apoptosis. For example, one of the caspases identified in humans was previously known as the interleukin-1 (IL-1 β) converting enzyme (ICE), a cysteine protease responsible for the processing of pro-IL-1β to the active cytokine.

Many caspases and proteins that interact with caspases possess domains of about 60 amino acids called a Caspase Recruitment Domain (CARD). Others have postulated that certain apoptotic proteins bind to each other via their CARDs and that different subtypes of CARDs may confer binding specificity, regulating the activity of various caspases, for example.

Innate immunity to bacterial pathogens relies on the specific sensing of pathogen-associated molecular patterns (PAMPs) by pattern-recognition molecules (PRMs). In mammals, Toll-like receptors (TLRs) represent the most extensively studied class of PRMs, which have been shown to sense various PAMPs, such as lipopolysaccharide (LPS), peptidoglycan (PGN), lipoproteins, double-stranded RNA and CpG DNA (Akira et al. (2001); Medzhitov (2001)). While TLRs are mainly expressed at the plasma membrane, it has been recently proposed that the Nod molecules, a family of intracellular proteins including Nod1/CARD4 and Nod2/CARD15, could represent a new group of PRMs that sense bacterial products within the cytoplasmic compartment, thus allowing to detect the presence of intracellular invasive bacteria (Inohara et al. (1999); Bertin, et al. (1999); Inohara et al. (2001); Girardin et al. (2001); Ogura et al. (2001); Ogura et al. (2001); Hugot et al. (2001)).

The partial sequences (cDNA and protein) of Nod1, also named CARD4 for Caspase Recruitment Domain, have been disclosed in patent application Ser. No. 09/019,942, filed Feb. 6, 1998, and now granted as U.S. Pat. No. 6,033,855. Furthermore, Bertin et al. (1999) disclosed the entire amino acid sequence of CARD4 and one of its functions, already mentioned in the above patent: CARD4 coordinates NF-κB and apoptotic signaling pathway. Girardin et al. (2001) disclosed that CARD4/Nod1 mediates NF-κB activation by invasive Shigella flexneri. In this article, the interaction between S. flexneri LPS and CARD4 is especially studied.

Stimulation of Nod1/CARD-4 activity is desirable in situations in which CARD-4 is abnormally downregulated and/or in which increased CARD-4 activity is likely to have a beneficial effect. Conversely, inhibition of CARD-4 activity is desirable in situations in which CARD-4 is abnormally upregulated, e.g., in myocardial infarction, and/or in which decreased CARD-4 activity is likely to have a beneficial effect. Since CARD-4 may be involved in the processing of cytokines, inhibiting the activity or expression of CARD-4 may be beneficial in patients that have aberrant inflammation.

It has recently been shown that Nod1 senses the presence of the Gram-negative pathogen, Shigella flexneri, within the cytoplasmic compartment of epithelial cells (Girardin et al. (2001)), and it was hypothesized that the detected PAMP was LPS since commercial preparations of LPS were shown to activate Nod1 (Inohara et al. (2001)). However, as these LPS often contain bacterial cell wall contaminants, there is a need in the art for more detail on whether a particular molecular motif or motifs are actually detected by Nod1 and modulate Nod1 activity.

The Gram-negative bacterium Helicobacter pylori is present in the stomachs of approximately one half of the world's population and is, arguably, the single most important cause of gastro-duodenal disease in humans. The severity of H. pylori-related disease, however, varies greatly amongst infected individuals and appears to be influenced by both host and bacterial factors. In Western populations, H. pylori strains harboring a 40 kilobase DNA region, known as the “cag” pathogenicity island (cagPAI), were found to be more frequently associated with severe gastric inflammation, ulceration and an increased risk of gastric cancer²⁻⁴. From in vitro studies, it has been shown that only H. pylori strains harboring a functional cagPAI induced cytoskeletal modifications (“cell scattering”) and NF-κB-dependent pro-inflammatory responses in gastric epithelial cells (Fischer et al (2001); Segal et al. (1999); Selbach et al. (2002)).

The cagPAI was proposed to encode a type IV secretion apparatus (Covacci et al (1998)). By analogy with equivalent systems in other bacterial pathogens (Cascales et al. (2003)), it was suggested that the apparatus was likely to mediate the translocation of protein effector molecule(s) into its target host cell (Covacci et al (1998)). Definitive proof for this suggestion was recently provided in the form of cagPAI-mediated translocation of a protein, CagA, in gastric epithelial cells (Selbach (2002); Odenbreit (2000)). While CagA translocation, and its phosphorylation, are required for H. pylori-induced cell scattering (Segal et al. (1999); Selbach et al. (2003)), these events are dispensable for H. pylori induction of NF-κB activation in host cells (Fischer et al. (2001)). Mutagenesis of the full complement of cagPAI genes failed to identify an obvious candidate protein for this activity (Fischer et al. (2001); Selbach et al. (2002)). Thus, the precise mechanism by which this extracellular pathogen is able to induce pro-inflammatory responses in gastric epithelial cells has remained obscure.

Epithelial cells express several types of transmembrane pathogen recognition molecules belonging to the family of Toll-like receptors (TLRs) (Backhed et al. (2003); Cario et al. (2002)). These molecules, which play a fundamental role in host innate immune responses, are able to recognize conserved microbial components, including various products of Gram-negative bacteria, such as lipoproteins (TLR2), lipopolysaccharide (LPS; TLR4) and flagellin (TLR5) (Backhed et al. (2003); Cario et al. (2002)). From recent work, it appears that the major pathogen recognition molecule for sensing of Gram-negative bacteria, TLR4, does not play a significant role in epithelial cell responses to H. pylori (Bäckhed et al. (2003); Maeda et al. (2001); Smith et al. (2003)). Furthermore, the data concerning the contribution of TLR2 and/or TLR5 towards epithelial cell recognition of H. pylori have been contradictory (Maeda et al. (2001); Smith et al. (2003); Lee et al. (2003)). Given that a functional type IV secretion apparatus is required for H. pylori-induced NF-κB activation in gastric epithelial cells, the inventors reasoned that an intracellular receptor may be involved in recognition of an H. pylori product that is presented within the cells.

A new family of intracytoplasmic pathogen recognition molecules with homology to plant resistance proteins, has recently been described. As mentioned above, two members of this family, Nod1/CARD4 and Nod2/CARD15, were reported to respond to different motifs within peptidoglycan (PG), a component of bacterial cell walls (Chamaillard et al. (2003); Girardin et al. (2003); Girardin et al. (2003)). The reported role of Nod1 as an important intracellular sensor of Gram-negative bacteria in epithelial cells (Girardin et al. (2001)) led the inventors to investigate the involvement of this molecule in host recognition of H. pylori. Because H. pylori is an important cause of gastroduodenal disease, there is a need for identifying the mechanism of the innate immune response in epithelia, and for understanding and manipulating how epithelial cells discriminate between pathogenic and commensal bacteria.

### SUMMARY OF THE INVENTION

This invention aids in fulfilling these needs in the art. In one embodiment, this invention provides a method for identifying a compound, which modulates the interaction between Nod1 and a Gram-negative bacteria comprising:
(a) contacting a Nod1 expressing cell with a cagPAI-positive H. pylori in the presence of a compound;
(b) contacting a Nod1 expressing cell with a cagPAI-positive H. pylori in the absence of said compound; and
(c) detecting the activation of a pro-inflammatory factor and/or production of a pro-inflammatory cytokine or chemokine in (a) and/or (b);
wherein altered activation of a pro-inflammatory factor and/or production of a pro-inflammatory cytokine or chemokine in (a) and/or (b) indicates that said compound modulates the interaction between Nod1 and the Gram-negative bacteria.

In one embodiment, the pro-inflammatory factor is NF-κB and the cytokine or chemokine is an NF-κB-dependent cytokine or chemokine. In a particular embodiment, the NF-κB-dependent cytokine or chemokine is IL-8 or MIP-2. In another embodiment, the Nod1 expressing cell is an epithelial cell.

In one embodiment, the activation of a pro-inflammatory factor and/or production of a pro-inflammatory cytokine or chemokine in (a) is increased. In another embodiment, the activation of a pro-inflammatory factor and/or production of a pro-inflammatory cytokine or chemokine in (a) is decreased.

The invention further provides that detecting the activation of a pro-inflammatory factor and/or production of a pro-inflammatory cytokine or chemokine in (a) and/or (b) comprises detecting NF-κB activation. In one embodiment, NF-κB activation is detected by a bioluminescent signal.

The invention also provides for abrogating the activation of a pro-inflammatory factor and/or production of a pro-inflammatory cytokine or chemokine in (a) and/or (b) by treating said Nod1 expressing cell with siRNA against Nod1. In one embodiment, siRNA against Nod1 comprises the polynucleotide 5′-ACAACTTGCTGAAGAATGACT-3′ [SEQ ID NO: 1]. The invention also comprises for abrogating the activation of a pro-inflammatory factor and/or production of a pro-inflammatory cytokine or chemokine in (a) and/or (b) by treating said Nod1 expressing cell with dominant-negative Nod1.

The invention also provides for a method for identifying a compound, which modulates the interaction between Nod1 and a Gram-negative bacteria comprising:

(a) contacting a Nod1 expressing cell with a cagPAI-positive H. pylori in the presence of a compound;

(b) contacting a Nod1 expressing cell with a cagPAI-positive H. pylori in the absence of said compound;

(c) contacting a Nod1 expressing cell with a cagPAI-negative H. pylori in the presence of said compound;

(d) contacting a Nod1 expressing cell with a cagPAI-negative H. pylori in the absence of said compound; and

(e) detecting the activation of a pro-inflammatory factor and/or production of a pro-inflammatory cytokine or chemokine in (a), (b), (c), and (d);

wherein altered activation of a pro-inflammatory factor and/or production of a pro-inflammatory cytokine or chemokine (a) and/or (b) and/or (c) and/or (d) indicates that said compound modulates the interaction between Nod1 and the Gram-negative bacteria.

In one embodiment, the pro-inflammatory factor is NF-κB and said cytokine or chemokine is an NF-κB dependent cytokine or chemokine. NF-κB dependent cytokine or chemokine can be, for example, IL-8 or MIP-2.

The invention further provides for a method for detecting a dysfunction of the inflammatory and/or apoptosis pathway in which Nod1 is involved, comprising:

(a) bringing a cagPAI-positive H. pylori into contact with a cell in which the dysfunction of the inflammatory and/or apoptosis pathway in which Nod1 is involved, is suspected;

(b) bringing a cagPAI-negative H. pylori into contact with a cell in which the dysfunction of the inflammatory and/or apoptosis pathway in which Nod1 is involved, is suspected, and

(c) evaluating activation of a pro-inflammatory factor and/or production of a pro-inflammatory cytokine or chemokine in (a) and (b),

wherein similar levels of activation of a pro-inflammatory factor and/or production of a pro-inflammatory cytokine or chemokine in (a) and (b) indicates dysfunction of a molecule of the inflammatory and/or apoptosis pathway in which Nod1 is involved. In one embodiment, the cell is an epithelial cell.

In one embodiment, the pro-inflammatory factor is NF-κB and the cytokine or chemokine is an NF-κB dependent cytokine or chemokine. NF-κB dependent cytokine or chemokine can be, for example, IL-8 or MIP-2.

In another embodiment, the cell is an epithelial cell. In another embodiment, evaluating activation of a pro-inflammatory factor and/or production of a pro-inflammatory cytokine or chemokine in (a) and (b) comprises detecting NF-κB activation. In a particular embodiment, NF-κB activation is detected by a bioluminescent signal.

The invention also encompasses a method for inactivating Nod1 in a Nod1 expressing cell comprising administration of siRNA against Nod1 in an amount sufficient to cause inactivation of Nod1. In one embodiment, the siRNA against Nod1 comprises the polynucleotide sequence 5′-ACAACTTGCTGAAGAATGACT-3′ [SEQ ID NO: 1]. In another, the Nod1 expressing cell is an epithelial cell. In yet another, the Nod1 expressing cell is transfected with about 50-500 ng of a construct comprising a sequence specific for CARD in human nod1. In one embodiment, this construct comprises the polynucleotide sequence 5′-ACAACTTGCTGAAGAATGACT-3′ [SEQ ID NO: 1].

The invention also encompasses a method for assaying whether a Gram-negative bacteria is cagPAI-positive comprising the steps of:

(a) contacting a Gram negative bacteria with a cell line expressing Nod1;

(b) contacting a Gram negative bacteria with a cell line not expressing Nod1; and

(c) evaluating activation of a pro-inflammatory factor and/or production of a pro-inflammatory cytokine or chemokine in (a) and (b);

wherein altered activation of a pro-inflammatory factor and/or production of a pro-inflammatory cytokine or chemokine in (a) and/or (b) indicates that said Gram-negative bacteria is cagPAI-positive.

In one embodiment, the pro-inflammatory factor is NF-κB and said cytokine or chemokine is an NF-κB dependent cytokine or chemokine. In a particular embodiment, the NF-κB dependent cytokine or chemokine is IL-8 or MIP-2.

The invention also encompasses evaluating activation of a pro-inflammatory factor and/or production of a pro-inflammatory cytokine or chemokine in (a) and (b) comprising detecting NF-κB activation. In one embodiment, NF-κB activation is detected by a bioluminescent signal.

In another embodiment, altered activation of a pro-inflammatory factor and/or production of a pro-inflammatory cytokine or chemokine in (a) and/or (b) is abrogated by treatment of the cell line expressing Nod1 with siRNA against Nod1. For example, siRNA against Nod1 can comprise the polynucleotide 5′-ACAACTTGCTGAAGAATGACT-3′ [SEQ ID NO: 1].

In another, embodiment, the altered activation of a pro-inflammatory factor and/or production of a pro-inflammatory cytokine or chemokine in (a) and/or (b) is abrogated by treatment of said cell line expressing Nod1 with dominant-negative Nod1.

The invention also encompasses a method of inducing a pro-inflammatory response and/or apoptosis in a cell containing intracellular Nod1, wherein the method comprises contacting said cell with H. pylori cell-free PG, a fragment thereof, or a related molecule thereof.

In one embodiment, the cell is a mammalian cell. In another, the cell is a mammalian gastric epithelial cell.

In another embodiment, the H. pylori PG fragment is H. pylori MTP or a molecule related to H. pylori MTP. In yet another embodiment, the method comprises activating an NF-κB signaling pathway in said cells.

The invention also encompasses a composition that comprises a biologically acceptable carrier and a biologically effective amount of H. pylori PG, H. pylori MTP, or a molecule related to H. pylori MTP.

In one embodiment, the composition is administered in a therapeutically effective amount to a human or animal in need thereof as a method for preventing or treating abnormal level or rate of apoptotic cell death or inflammation. In another embodiment, the composition is administered in an effective amount to a human or animal in need thereof, as a method for preventing or treating a Gram-negative bacteria infection.

### BRIEF SUMMARY OF THE DRAWINGS

This invention will be described in greater detail with reference to the drawings in which:

FIG. 1 shows that the Nod1 pathway is stimulated by commercial LPS and Gram-negative bacterial PGNs.

FIG. 2 is a characterization of the PGN motif detected by Nod1.

FIG. 3 shows the results of intracellular detection of Gram-negative, but not Gram-positive bacterial products, in epithelial cells through Nod1/Rip2, but not MyD88.

FIG. 4 depicts the experimental procedure followed to prepare highly purified PGNs from Gram-negative or Gram-positive bacteria.

FIG. 5 is a schematic representation of the muropeptides derived from PGNs of various bacteria. a, muropeptide found in Gram-negative bacteria, containing a mesoDAP amino acid. b, muropeptide found in some Gram-postivie bacteria such as B. subtilis. Instead of the mesoDAP, an amidated-DAP is found. c, muropeptide found in most Gram-positive bacteria, containing a L-lysine as third amino acid. Note that the only difference between these muropeptides take place at the third amino acid position.

FIG. 6 depicts the results of the relative PGN content of bacterial extracts prepared. NF-_{K}B activation was assessed by translocation of the NF-kB p65 subunit from the cytosol to the nucleus in microinjected cells. a. We aimed to determine the relative PGN content of the bacterial extracts prepared. To this end, TLR2-overexpressing HEK293 cells were stimulated by extracellular addition of bacterial extracts treated or not with proteinase K and boiling, which affects lipoprotein activity. In this way, we could analyze the relative contribution of bacterial lipoprotein versus PGN towards TLR2 stimulation. We observed that bacterial extracts from Gram-negative bacteria displayed much less PGN activity than extracts from Gram-positive bacteria cultured to the same optical density (FIG. S3a). This finding is not surprising since it is well known that Gram-negative bacteria contain less PGN than Gram-positive bacteria and also recycle PGN to a higher extend during growth. b. Bacterial extracts from S. flexneri and S. aureus were micro-injected together with FITC-Dextran into Caco-2 epithelial cells. After 30 minutes, the cells were fixed and analyzed for NF-_{K}B activation by immunofluorescence. NF-κB activation was assessed by translocation of the NF-_{K}B p65 subunit from the cytosol to the nucleus in microinjected cells. Activation of NF-_{K}B was observed only in Caco-2 cells microinjected with bacterial extracts from Gram-negative bacteria. We also observed the lack of NF-_{K}B activation in Caco-2 cells following extracellular addition of bacterial extracts, suggesting that these cells do not display a functional endogenous TLR sensing system.

FIG. 7 shows that Nod1 and Nod2 are expressed in HeLa and HEK 293 epithelial cells. Nod1 and Nod2 are expressed in HeLa and HEK 293 epithelial cells. a. Protein extracts (2·10⁴ cells/lane) from HeLa and HEK 293 cells were directly analyzed by western blot for endogenous expression of Nod1 and Nod2, using specific polyclonal antibodies raised against human forms of Nod1 and Nod2, respectively (see Methods Section). Note that endogenous expressions of Nod1 and Nod2 remained undetectable using this technique, and therefore protein extracts from HEK 293 transiently transfected with expression vectors for Nod1 (HEK/Nod1) and Nod2 (HEK/Nod2) were analyzed in parallel in order to show the specificity of the antibodies. b. Protein extracts (10⁷ cells/lane) from HeLa and HEK 293 cells were immunoprecipitated using Nod1, Nod2 or control IgG antibodies prior to analysis by western blot as indicated. This immunoprecipitation technique was required to detect the presence of the endogenous forms of Nod1 and Nod2, since it allowed for the concentration of Nod1 and Nod2 present in cell extracts by ˜500 fold.

FIG. 8A shows that detection of peptidoglycan by Nod1 does not require a cyclic sugar moiety.

FIG. 8B shows that Nod1 detects meso-DAP-containing GM-tripeptide, but not amidated-DAP-containing GM-tripeptide.

FIG. 8C shows that Nod1 detects TriDAP.

FIG. 8D shows that Nod1 detects UDP-MurTriDAP and MurTriDAP.

FIG. 9 shows that HEK293 cells express nod1 mRNA. Expression of nod1 in HEK293, HeLa and AGS epithelial cells was assessed by RT-PCR. The quantities of total RNA were standardized for each cell line by performing PCR using oligonucleotides specific for β-actin. The arrows indicate the respective molecular sizes (in base pairs, bp) of the amplified products.

FIG. 10 shows that Nod1 mediates epithelial cell responses to cagPAI-positive H. pylori bacteria. a, NF-κB-dependent luciferase reporter activity in HEK293 cells stimulated with bacterial strains possessing either functional (H. pylori strains 245, 251, 251 cagA and 256) or non-functional cagPAI secretion apparatus (H. pylori 251 cagMor SS1), or in which cagPAI genes were absent (H. felis). Cells transfected with the vector alone (pCDNA3) were used as a negative control. b, NF-κB reporter activity in HEK293 cells that had been co-transfected with increasing amounts (50-500 ng) of a dominant-negative construct (DN-Nod1) then stimulated with cagPAI-positive H. pylori strains 251, 256, 26695 or N6. Similar results were obtained for H. pylori strains 251 cagA and NCTC11637 (data not shown). The total quantity of DNA was standardized throughout using vector (pCDNA3) DNA alone. c, NF-κB reporter activity in DN-Nod1-transfected HEK293 cells following TNF stimulation. d, Western blot analysis of Nod1 and β-tubulin production in Nod1 over-expressing HEK293 cells following transfection (48 h) with increasing concentrations (50-500 ng) of Nod1siRNA (lane 1) or EGFPsiRNA (lane 2). As a control, cells were transfected with pCDNA3 (500 ng). e, NF-κB-dependent luciferase reporter activity in HEK293 cells that had been transfected (72 h) with either Nod1siRNA, EGFPsiRNA or pCDNA3 alone (50 ng) then stimulated with H. pylori 251 or 251 cagM bacteria. Similar results were obtained for cells stimulated with H. pylori strains 256 and 26695, respectively (data not shown). Reporter assay data correspond to the mean±SEM (triplicate determinations) and are representative of 3 or more independent experiments.

FIG. 11 shows that H. pylori PG induces pro-inflammatory responses in epithelial cells. a, NF-κB-dependent luciferase reporter activity in Nod1 co-transfected cells, in response to intracellular stimulation with H. pylori PG (1 μg), or LPS (10 μg) (P=0.03). b, PG muropeptides from H. pylori were separated by HPLC (inset), and then each fraction was tested for its effect on NF-κB-dependent luciferase reporter activity in HEK293 cells. Fraction 1, corresponding to the GM-tripeptide, specifically activated Nod1-dependent NF-κB activity in the cells. c, NF-κB-dependent luciferase reporter activity in HEK293 cells and d, IL-8 secretion in AGS cells following stimulation with either H. pylori 26695 or 26695slt bacteria (P=0.004; P=0.0009, respectively). Data correspond to the mean±SEM (triplicate determinations) and are representative of 3 or more independent experiments.

FIG. 12 shows that a functional cagPAI is required for H. pylori delivery of PG to epithelial cells. AGS cells were co-cultured with H. pylori 26695 and 251 bacteria. Specific incorporation of ³H-DAP label into PG was achieved by lysA gene inactivation in wild-type bacteria (see text). a, Radioactivity levels (counts per min, CPM) per mm² in cells co-cultured with bacteria possessing either functional (lysA) or non-functional (lysAcagM; lysA heat-killed, HK) cagPAI. Data are expressed as percentage values of the means in CPM per mm² (calculated from duplicate or triplicate readings), from two independent experiments. b-d, The presence of tritiated PG in AGS co-cultures was visualized by brightfield photomicrography, performed on slides that had been immersed in nuclear emulsion. Radioactive label is revealed by the deposition of silver particles. Heavy (panel b, large arrow heads) and sparse (panels c and d, small arrows) levels of silver deposition, corresponding to ³H-labeled PG, in cells co-cultured with (b) H. pylori 26695 lysA, (c) H. pylori 26695 lysAcagM or (d) lysA HK bacteria. Slides were counter-stained with Giemsa stain. Scale bar, 50 μm.

FIG. 13 shows dual immunohistochemical and silver labeling of H. pylori co-cultured AGS cells. AGS cells were either (a) left untreated, or were co-cultured for 4 h with (b) H. pylori 251 lysA, (c) H. pylori 251 lysA cagM, or (d) HK H. pylori 251 lysA. An anti-H. pylori whole cell antibody reacted with adherent bacteria at the external surfaces of the cells (arrow heads), and cross-reacted non-specifically with cell membranes (arrows). Heavy deposits of silver (panel b, asterisk) were only observed in AGS cells that had been incubated with H. pylori 251 lysA bacteria. Slides were counter-stained with Hemotoxylin stain. Scale bar, 100 μm.

FIG. 14 shows that Nod1^{-/-} mice have a heightened susceptibility to cagPAI-positive H. pylori bacteria. Nod1^{-/-} and WT mice were inoculated per os, and sacrificed at days 7 and/or 30 post-inoculation (p. i.). Bacterial loads for mice inoculated with (a) H. pylori 256 (n=5 mice per group), (b) B128 wild-type or B128cagM strains (n=3-7 mice per group), or (d) H. felis (N=10 mice per group). H. pyloribacterial loads in mice were determined by quantitative culture of gastric tissue samples, and are representative of two independent experiments. Since H. felis does not readily form colonies on culture plates, colonization levels were determined by “blind” scoring of Giemsa-stained histological sections. The proportion of fields with a given score (0, 1, 2 or 3) are presented for each group of mice (n=10 mice per group). Scores corresponded to high power fields in which none, 1-10, 10-100 or >100 bacteria, respectively, were recorded (≧50 fields graded per tissue sample). Each box-plot presents the median (central line within each box), the 25^{th} and 75^{th} percentile values (box ends), and the 10^{th} and 90^{th} percentile values (error bars).

FIGS. 15(A) and (B) show that primary gastric epithelial cells from Nod1^{-/-} mice produce lower amounts of MIP-2 in response to stimulation by H. pylori bacteria. Gastric epithelial cells were isolated from Nod1^{-/-} and Nod1^{+/+} mice and analyzed by (a) immunofluorescence with anti-actin, NF-κB p65 and cytokeratin antibodies, and (b) ELISA for MIP-2 production. MIP-2 data correspond to the mean±SEM (triplicate determinations) for one experiment. MIP-2 production was reduced by 25-87% and 44-87%, respectively, in Nod1^{-/-} cells stimulated with H. pylori B128 and 256 strains, when compared to Nod1^{+/+} cells (n=4-5 independent experiments).

### DETAILED DESCRIPTION OF THE INVENTION

As used herein, MTP means muramyl tripeptide from the peptidoglycan of the cell wall of a Gram-negative bacteria, that is GlcNAc-MurNAc-L-Ala-D-Glu-mesoDAP or meso-DAP containing GM-tripeptide.

As used herein, the terms “a molecule related to MTP” means a molecule or a compound having activity that is agonist or antagonist to the activity of MTP on Nod1. Molecules related to MTP comprise, but are not limited to, MTP the tripeptide L-Ala-D-Glu-mesoDAP, biologically active derivatives of MTP, such as, for example, the peptide fraction of MTP that is the three amino acids without the sugar moieties, MTP without GlcNAc, MTP with non-cyclized sugar, peptidomimetics, and molecules having activity that is antagonist to the one of MTP on Nod1.

The activity of the molecule related to MTP can be evaluated by the tests disclosed in the Examples: Evaluation of NF-κB activation or of IL-8 production for instance. A molecule having activity as agonist to MTP activity on Nod1 increases NF-κB activation or IL-8 production. A molecule having activity as antagonist to MTP activity on Nod1 decreases NF-κB activation or IL-8 production.

As used herein, the terms “biologically active derivatives” refers to function-conservative variants, homologous proteins or peptides and peptidomimetics, as well as a hormone, an antibody or a synthetic compound, (i.e., either a peptidic or non-peptidic molecule) that preferably retains the binding specificity and/or physiological activity of the parent peptide, as defined herein.

Also part of the invention are preferred peptidomimetics retaining the binding specificity and/or physiological activity of the parent peptide, as described herein and that they are positive in a test of activity as disclosed for testing one.

As used herein, “peptidomimetic” is an organic molecule that mimics some properties of peptides, preferably their binding specificity and/or physiological activity. Preferred peptidomimetics are obtained by structural modification of peptides according to the invention, preferably using unnatural amino acids, D. amino acid instead of L. amino acid, conformational restraints, isosteric replacement, cyclization, or other modifications. Other preferred modifications include without limitation, those in which one or more amide bond is replaced by a non-amide bond, and/or one or more amino acid side chain is replaced by a different chemical moiety, or one or more of the N-terminus, the C-terminus or one or more side chain is protected by a protecting group, and/or double bonds and/or cyclization and/or stereospecificity is introduced into the amino acid chain to increase rigidity and/or binding affinity.

Modifications can also be made for preparing molecules having activity that is antagonist to the one of Nod1.

While the role of Toll-like receptors in extracellular bacterial sensing has been investigated intensively, intracellular detection of bacteria through Nod molecules remains largely uncharacterized. This invention shows that Nod1 detects specifically a unique diaminopimelate-containing GlcNAc-MurNAc tripeptide motif found in Gram-negative bacterial peptidoglycan, resulting in activation of the NF-κB pathway. Moreover, this invention shows, in epithelial cells, which represent the first line of defense against invasive pathogens, that Nod1 is indispensable for intracellular Gram-negative bacterial sensing. Nod1 represents so far the first example of a pathogen-recognition molecule that specifically senses Gram-negative bacterial peptidoglycan.

Furthermore, this invention shows that Nod1 specifically senses MTP and more specially the peptide moiety of the GlcNAc-MurNAc tripeptide.

In the present invention, the inventors show that LPS was a contaminant in the previous studies and that Nod1 detects specifically a unique motif found in the peptidoglycan of Gram-negative bacteria: a muramyl tripeptide carrying at its third position a diaminopimelic amino acid.

Numerous publications are directed to MDP (muramyl dipeptide) and its adjuvant property. There are also publications about the adjuvant properties of MTP (muramyl tripeptide), which are generally directed to MTP-PE that is to muramyl tripeptide phosphatidylethanolamine. A first patent, FR 2160326, filed Nov. 19, 1971, concerns a process for preparing a soluble agent having an adjuvant activity, wherein the soluble agent is extracted from mycobacteria or Nocardia cell walls. FR 2248025, filed on Oct. 23, 1973, and is an addition to FR 2160326 and discloses the discovery that the adjuvant activity of this soluble agent comes from soluble fragments of peptidoglycans of the cell wall and concerns specific muramyl peptides. The U.S. equivalent is U.S. Pat. No. 4,186,194. The subject invention was made as follows.

The tripeptide according to the U.S. Pat. No. 4,186,194 contains always sugar moieties.

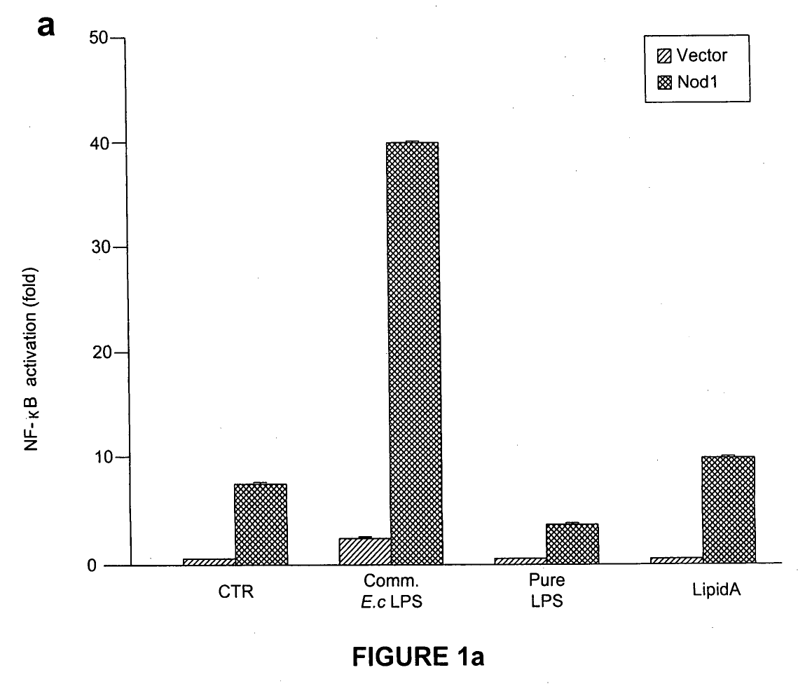

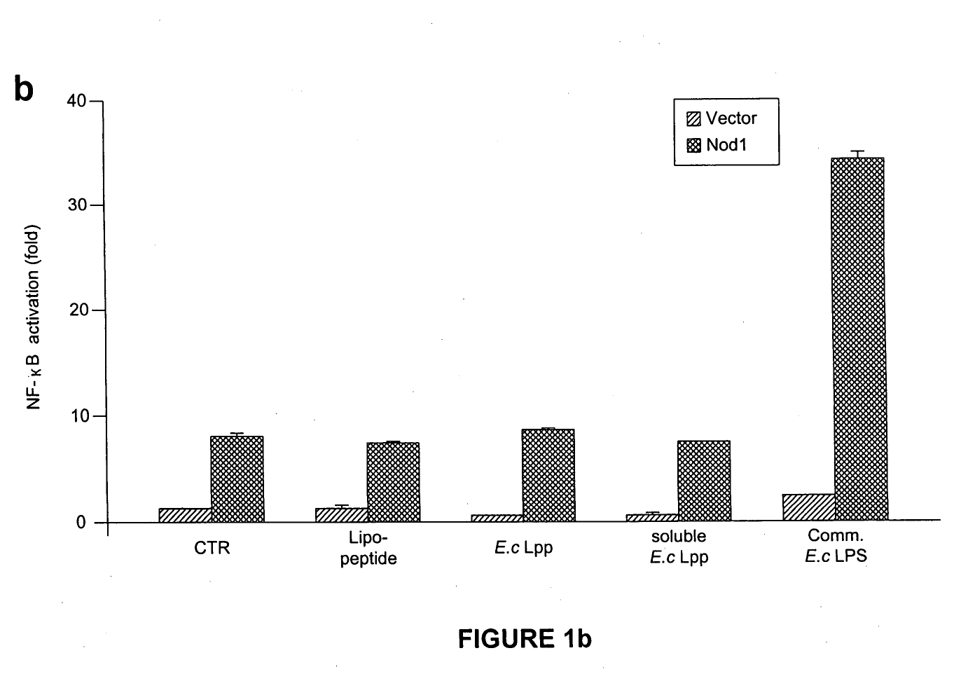

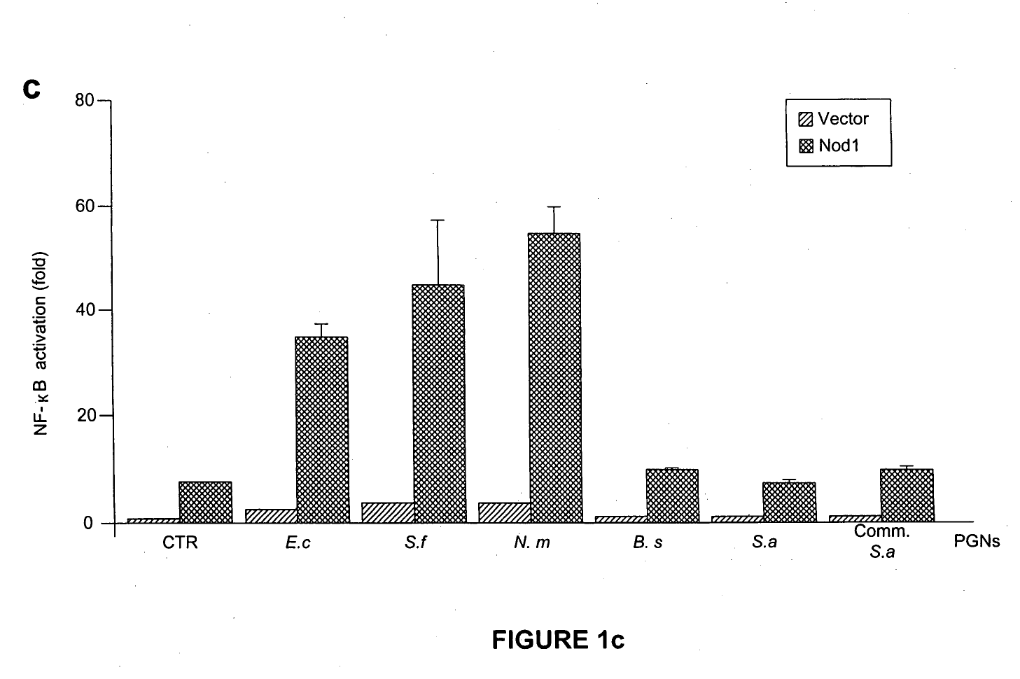

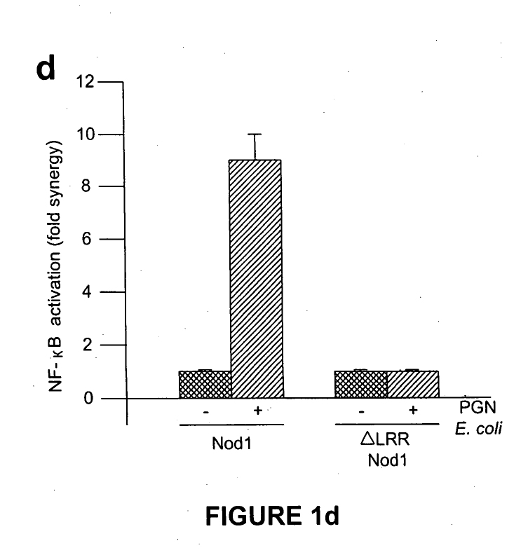

Addition of a commercial preparation of Escherichia coli LPS (10 μg) to Nod1-overexpressing HEK293 cells could potentiate by ˜5 fold the level of Nod1-dependent NF-κB activation (FIG. 1a). On the contrary, highly purified E. coli LPS (10 μg) or lipid A (10 μg) could not stimulate the Nod1 pathway (FIG. 1a), although they could efficiently activate macrophages. These findings show that a contaminant present in commercial LPS preparations is likely responsible for Nod1 activation. Therefore, one aim was to identify the nature of this contaminant. Lipoproteins have been recently identified as the major contaminants of LPS preparations responsible for TLR2 signaling following stimulation with commercial E. coli LPS (Lee et al. (2002)). However, it was not possible to stimulate the Nod1 pathway by addition of either synthetic lipopeptide or Lpp, the most abundant lipoprotein in E. coli (FIG. 1b). Moreover, boiling or proteinase K treatment of the commercial LPS was not sufficient to abolish Nod1 signaling.

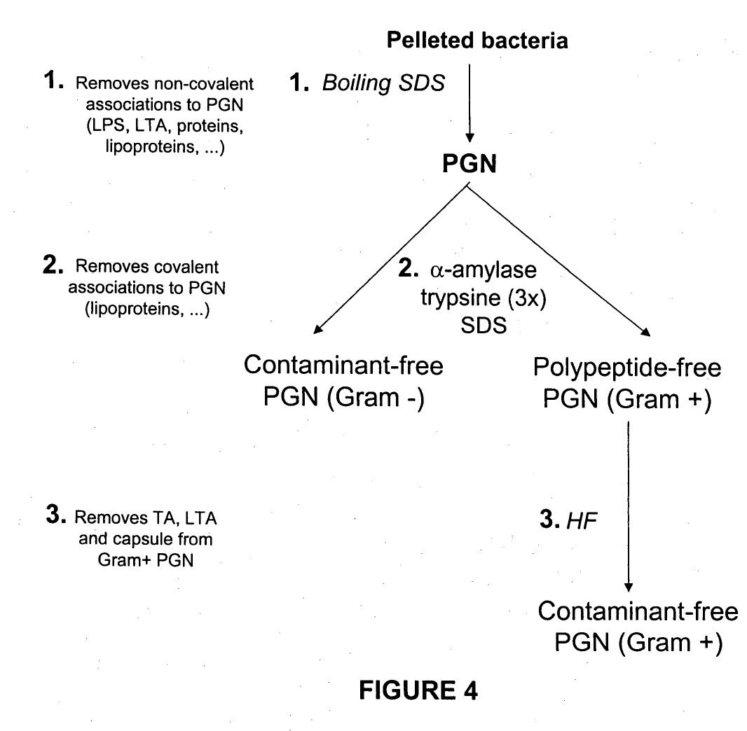

The next aim was to address the potential role of PGN in stimulating the Nod1 signaling pathway. Therefore, PGNs from E. coli, S. flexneri, Neisseria meningitides, Bacillus subtilis and Staphylococcus aureus were purified according to experimental procedures specifically designed for Gram-positive or Gram-negative bacteria (de Jonge et al. (1992); Glauner et al., (1988)). The harsh purification steps used to purify these PGNs eliminate the possible contaminants (FIG. 4). Strikingly, it was observed that PGN preparations from Gram-negative bacteria could stimulate the Nod1 pathway, while the two Gram-positive PGN preparations tested here could not (FIG. 1c). Moreover, by using a mutant form of Nod1 that lacks the C-terminal leucine-rich repeats (LRRs), it was observed that Nod1 LRRs play a critical role in the sensing of Gram-negative PGN (FIG. 1d). Therefore, these results strongly suggested that Nod1 is an intracellular PRM that specifically senses Gram-negative PGN through the LRR domain.

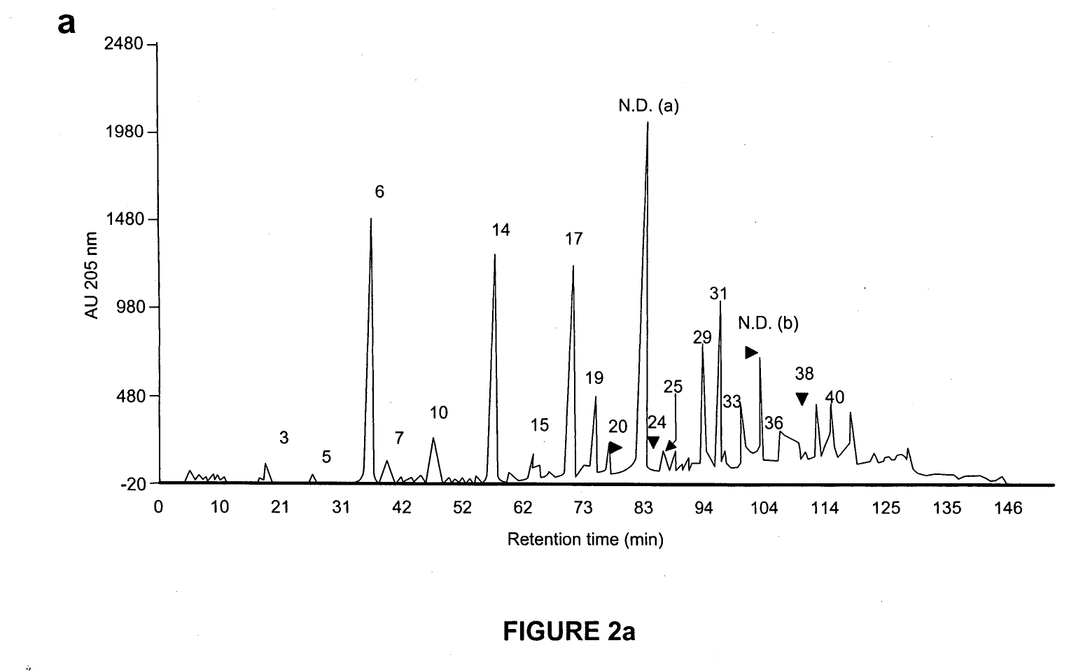

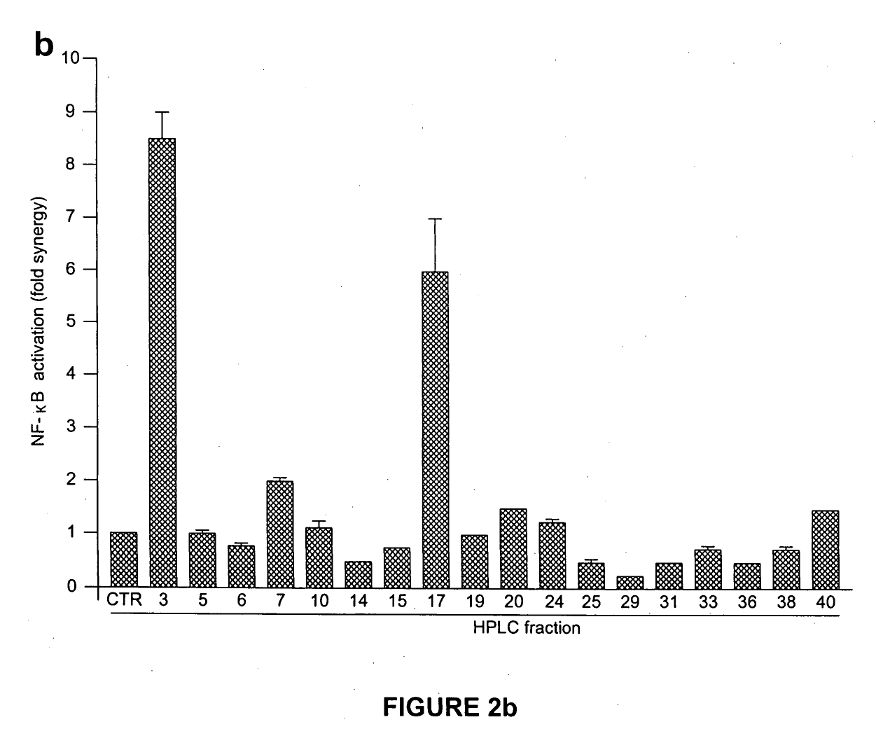

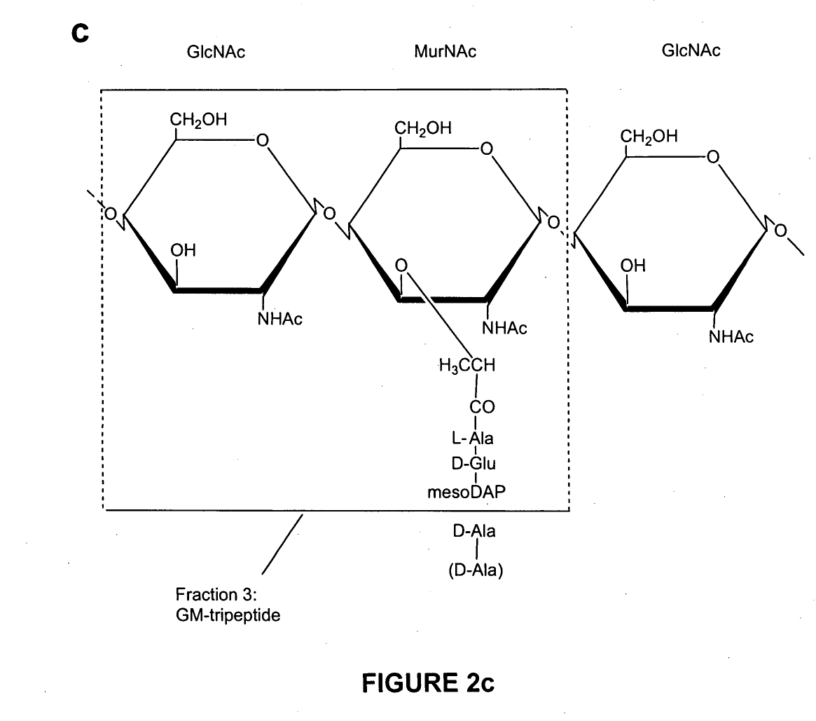

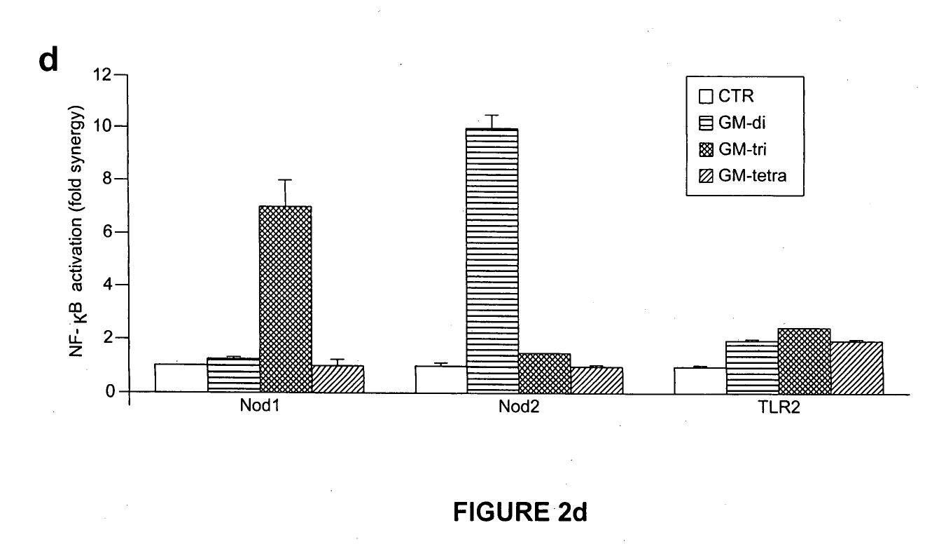

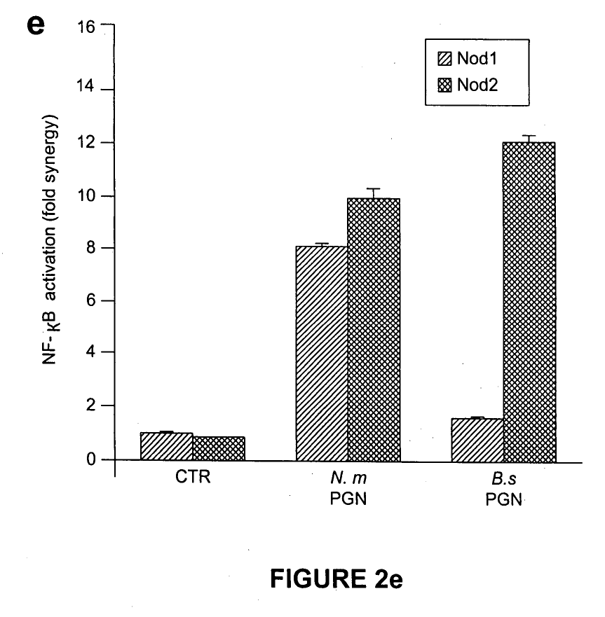

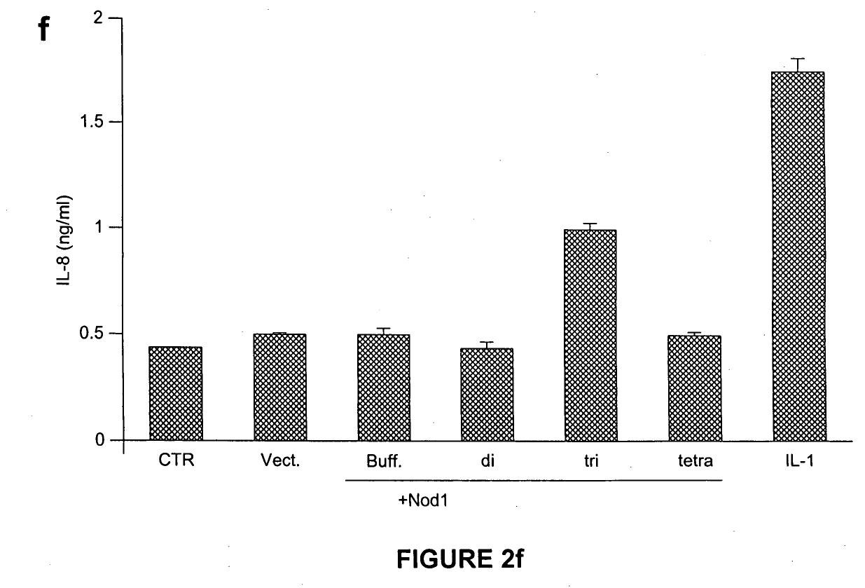

In order to identify the minimal PGN motif detected by Nod1, muropeptides from N. meningitidis were analyzed by reverse-phase HPLC after PGN digestion with a muramidase. Indeed, the major PGN fragments naturally released by Gram-negative bacteria are muropeptides (Höltje (1988); Blackburn et al. (2001)). This analysis allowed for the separation of muropeptides according to the number of amino acids of the peptidic chain linked to the amino sugars, the degree of polymerization of the peptidic chain or natural modifications such as O-acetylation or dehydration of the amino sugars (FIG. 2a).

Individual muropeptides were collected and tested for their ability to activate the Nod1 pathway. Surprisingly, only two fractions (3 and 17) contained muropeptides able to activate Nod1 (FIG. 2b). Mass spectroscopy analysis revealed that fraction 3 is a muropeptide with a molecular mass of 893 m/z and the active molecule in fraction 17 is a muropeptide of 873 m/z. The 893 m/z molecule is consistent with a reduced muropeptide Nacetylglucosamine (GlcNAc or “G”) β-1,4 linked to N-acetylmuramic acid (MurNAc or “M”), substituted with a tripeptide group (FIG. 2c), while the 873 m/z molecule corresponds to the same muropeptide naturally dehydrated on the MurNAc moiety (anhydro-MurNAc). The tripeptide group substituted on the MurNAc, L-Ala-D-Glu-mesoDAP (where Ala is Alanine, Glu is Glutamine and DAP is diaminopimelate), is therefore the same in the fractions 3 and 17. HPLC analyses and biological assays were carried out on muropeptides isolated from S. flexneri with similar findings.

To gain more insight into the molecular pattern sensed by Nod1, a comparison was made of the activation of the Nod1 pathway by GM-dipeptide, GM-tripeptide and GM-tetrapeptide. Equivalent amounts (10 ng) of GM-dipeptide, GM-tripeptide (from fraction 3) and GM-tetrapeptide (fraction 6) were tested for their ability to activate the Nod1 pathway. It was observed that Nod1 specifically detects GM-tripeptide but not GM-dipeptide nor GM-tetrapeptide (FIG. 2d). Since Nod1 is closely related to Nod2, these PGN products were also tested for Nod2 detection. Previous findings and those of Inohara et al. had shown that Nod2 recognizes M-dipeptide (Girardin, et al. (2003); Inohara et al. (2003)). Strikingly, it was discovered that Nod2 detects GM-dipeptide in addition to M-dipeptide but not GM-tripeptide or GM-tetrapeptide (FIG. 2d), suggesting that these two Nod molecules both sense PGN but require distinct molecular motifs to achieve detection. It was also observed that TLR2 could not detect any of the muropeptides tested (FIG. 2d).

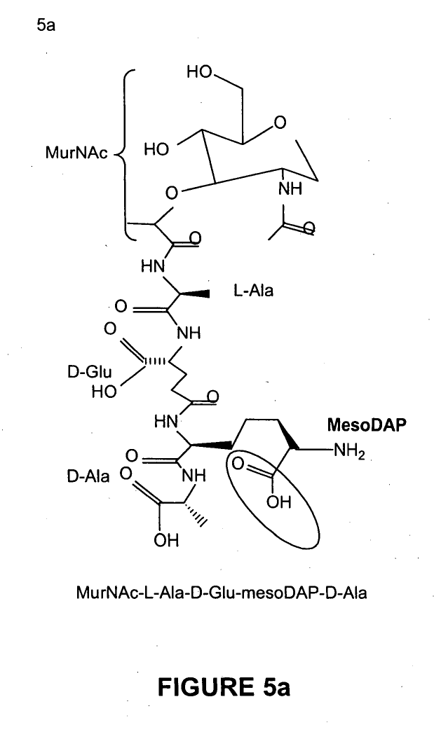

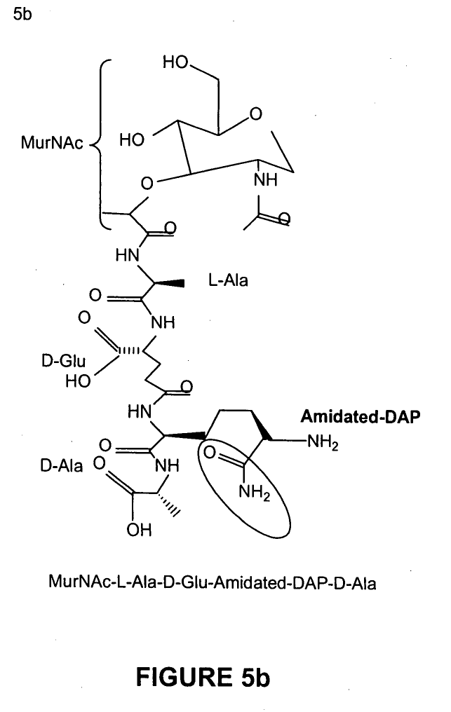

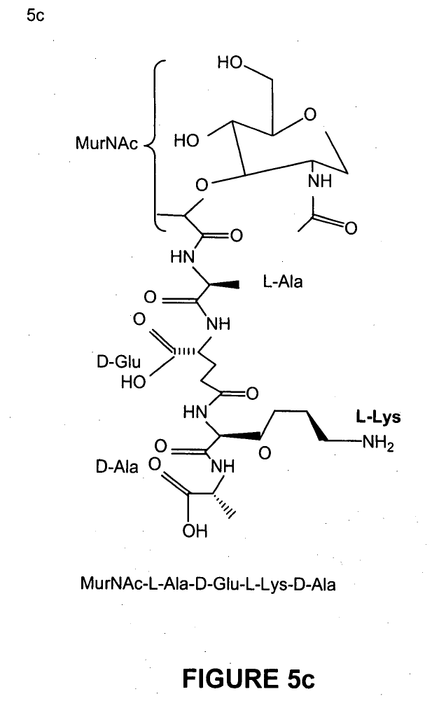

The PGN motif sensed by Nod2, GM-dipeptide, is found in all bacteria, suggesting that Nod2 is a general sensor of PGN degradation products (Girardin, et al. (2003); Inohara et al. (2003)). In contrast, the additional requirement of mesoDAP for Nod1 sensing explains why Nod1 detects only those PGNs purified from Gram-negative bacteria (see FIG. 1c) as compared to the Gram-positive PGNs tested. Indeed, while S. aureus PGN contains lysine instead of mesoDAP, B. subtilis contains amidated-diaminopimelate (FIG. 5). Furthermore, S. aureus PGN does not have any detectable amounts of GM-tripeptide (de Jonge et al. (1992)). In addition, these findings also indicate that the Nod1 sensing system requires an exposed mesoDAP since the GM-tetrapeptide, which also contains mesoDAP, is not sensed by Nod1. It was next observed that Nod2, which does not require mesoDAP to achieve PGN detection, could sense both B. subtilis and N. meningitidis PGNs (FIG. 2e). In contrast, even if the B. subtilis PGN shares some structural similarities with mesoDAP containing PGNs (FIG. 5), Nod1 does not detect B. subtilis PGN (FIG. 2e, also shown in FIG. 1c). In addition to NF-κB activation, it was found that sensing of the GM-tripeptide by Nod1 also leads to the production of the pro-inflammatory chemokine, interleukin-8 (FIG. 2f), which is one of the major cytokines produced by epithelial cells infected with Gram-negative bacteria (Eckmann et al. (2000); Pedron et al. (submitted)). Taken together, these results show that for Nod1-dependent activation of NF-κB and IL-8, the PGN structural requirements include GM linked to a tripeptide and a terminal mesoDAP amino acid in which both carboxy groups play a major role.

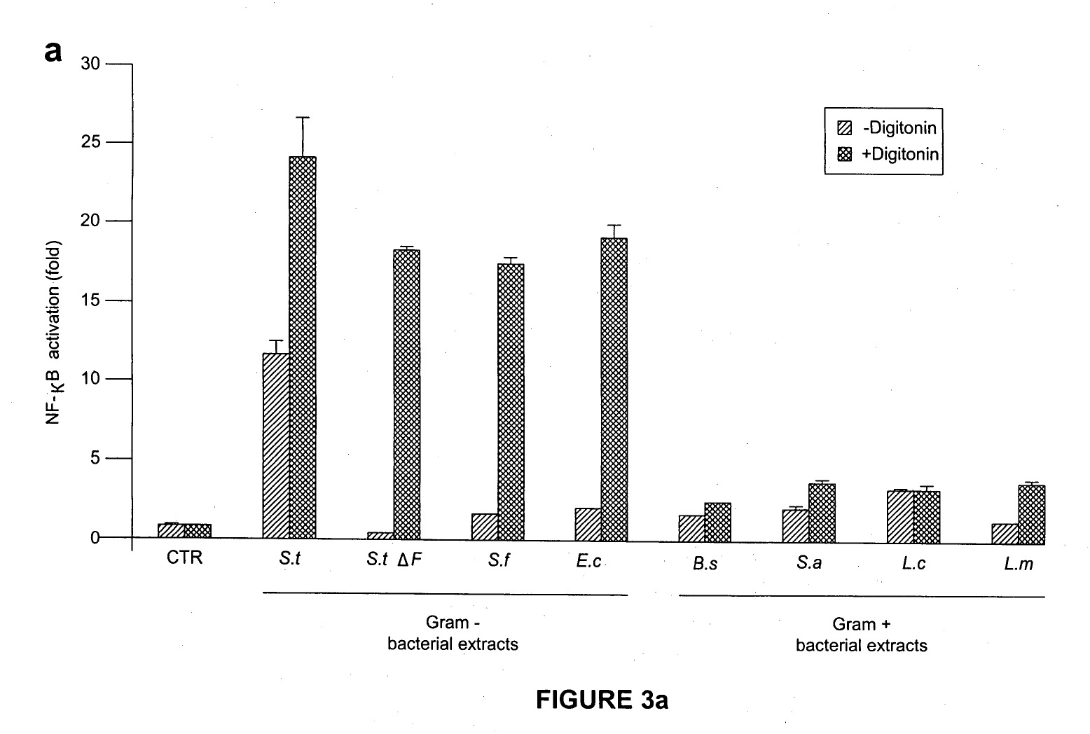

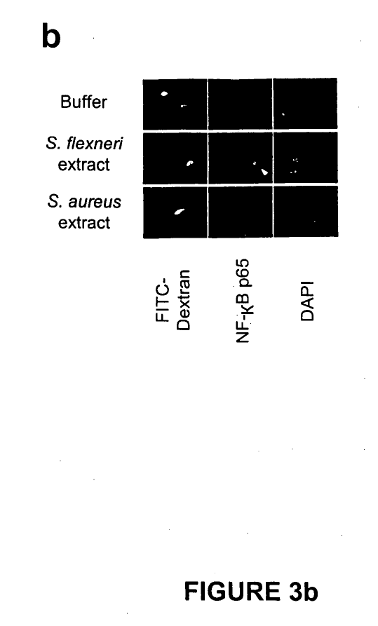

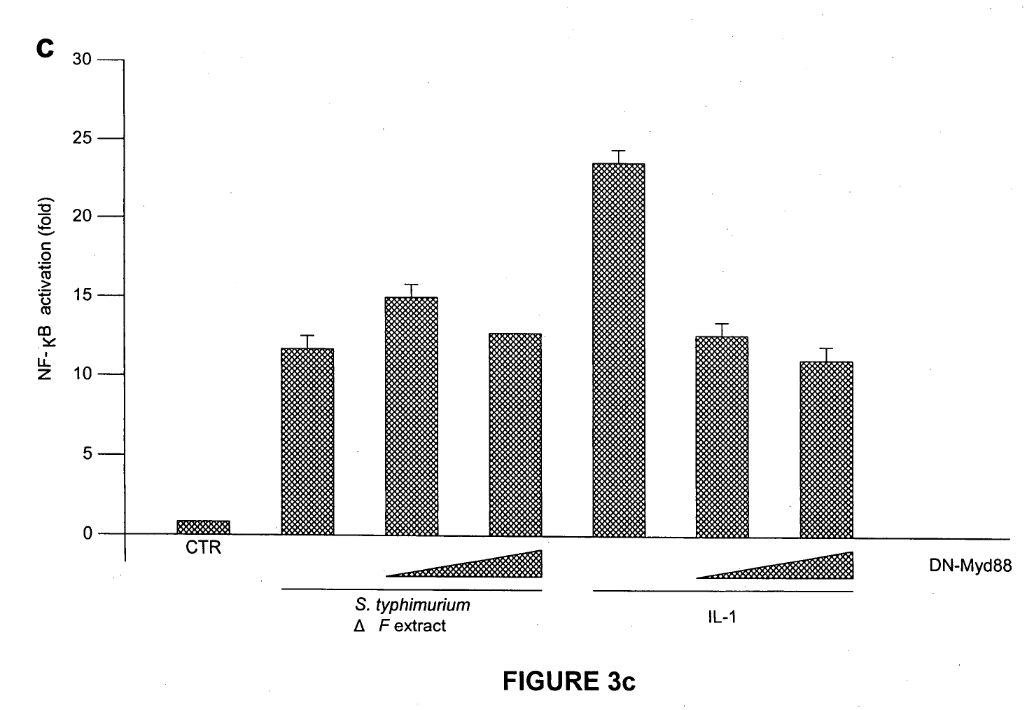

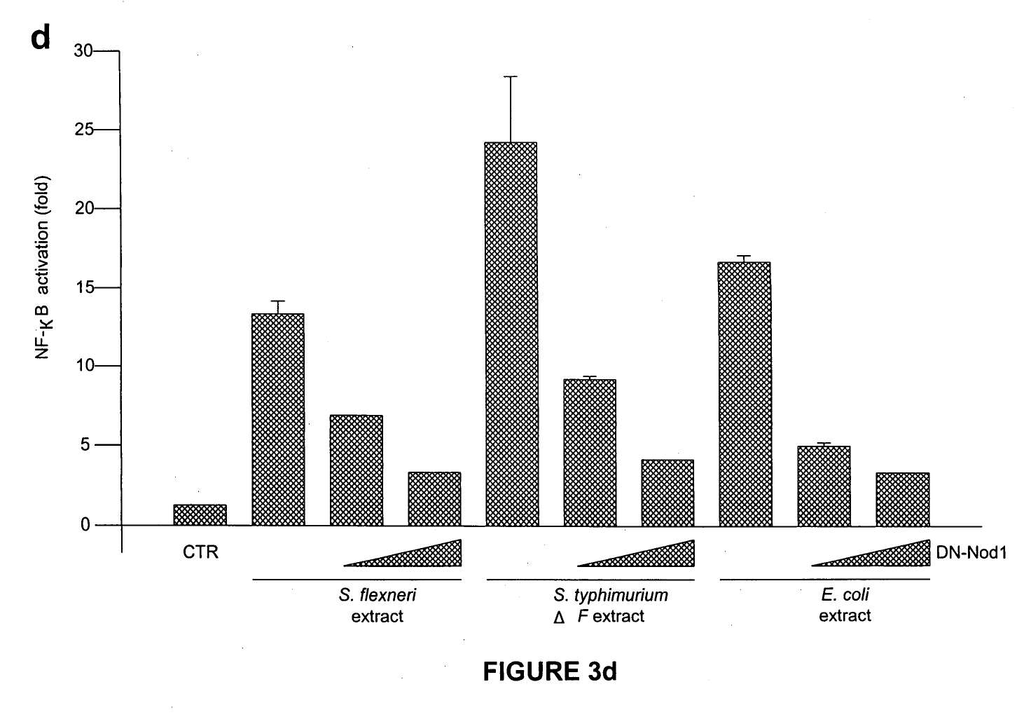

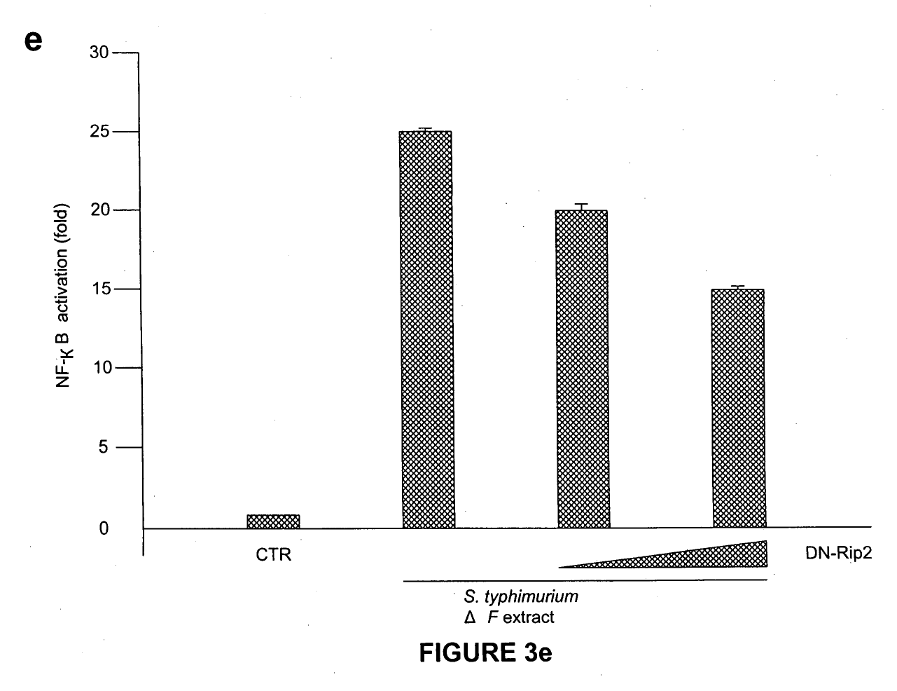

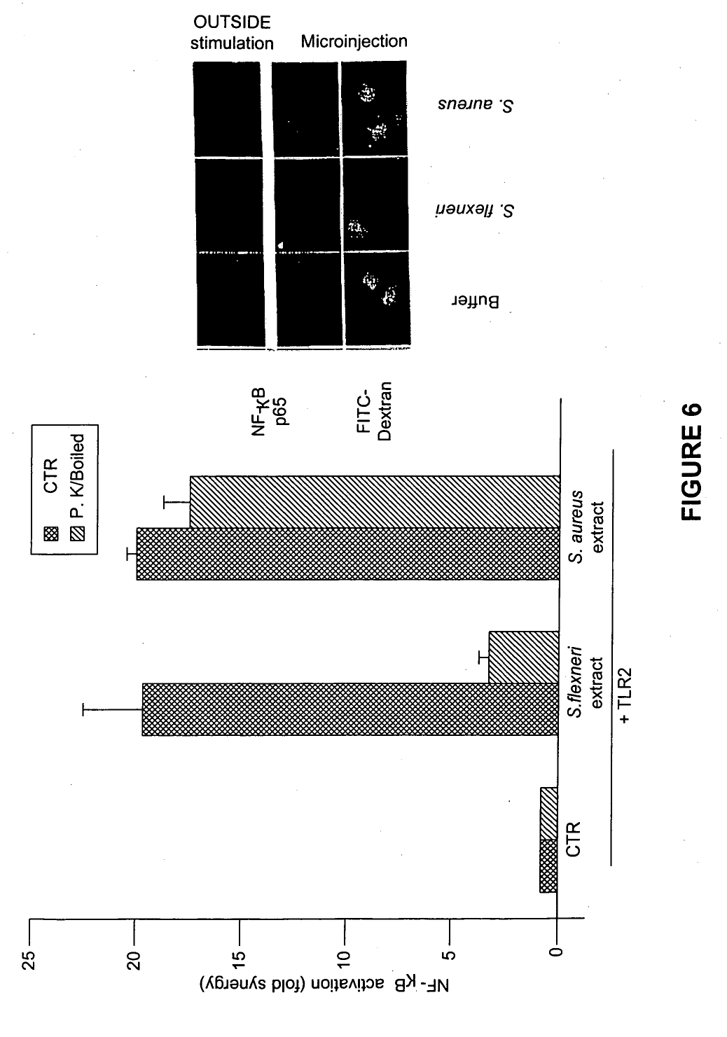

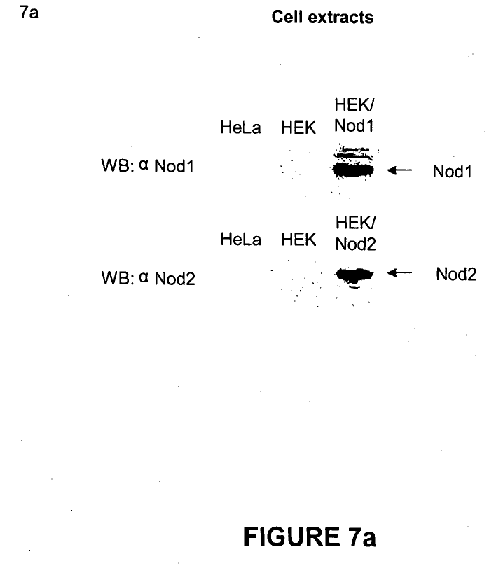

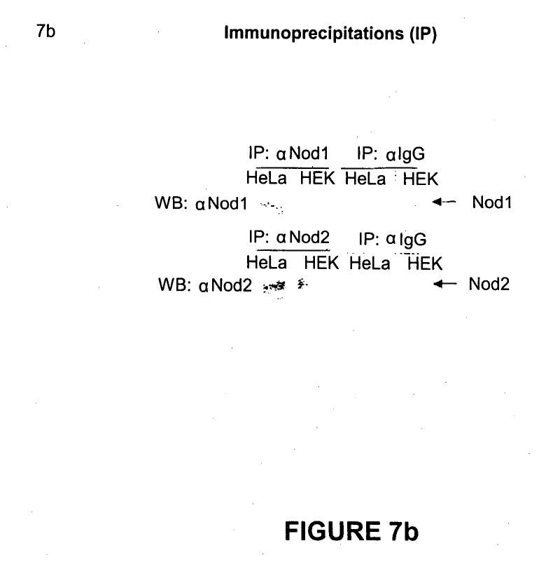

Next, it was of interest to determine the contribution of PGN detection by Nod1 in the context of intracellular bacterial sensing by epithelial cells. Indeed, previous studies stressed the pivotal role of these cells as the first line of defense against bacterial pathogens at mucosal surfaces. First, extracts were prepared from various Gram-negative or Gram-positive bacteria and determined the relative PGN content of these extracts (FIG. 6a). Then, these bacterial extracts were added extracellularly and showed that they were unable to activate NF-κB in HEK293 epithelial cells (FIG. 3a), confirming that these cells do not display an endogenous TLR2/4 sensing system. The only exception was Salmonella typhimurium extract; in this case NF-κB activation is likely to involve TLR5 (Hayashi, et al. (2001)), since extracts from a flagellin-deficient S. typhimurium strain were unable to stimulate the NF-κB pathway (FIG. 3a). A digitonin-based permeabilization technique was then used to elicit entry of bacterial products into the cytoplasm allowing a direct comparison of the ability of bacterial products from either invasive or non-invasive bacteria to activate the NF-κB pathway. It was observed that extracts from a number of Gram-negative bacteria were able to stimulate the NF-κB pathway while those from the four Gram-positive bacteria were not (FIG. 3a). The specific activation of NF-κB by only Gram-negative extracts was confirmed in two other epithelial cell lines (HeLa and Caco-2) by microinjection of bacterial products from either S. flexneri or S. aureus, followed by detection of the NF-κB p65 subunit nuclear translocation by immunofluorescence (FIG. 3b and FIG. 6b).

Therefore, these data show that epithelial cells sense Gram-negative but not Gram-positive bacterial products when presented to the cytoplasmic compartment. These findings are consistent with the fact that the released PGN motifs from the Gram-negative bacteria tested here all contain GM-tripeptide with a terminal mesoDAP, and that released Gram-positive bacterial PGN products lack this structure. In the case of L. monocytogenes, the PGN contains mesoDAP; however, the PGN degradation products have not yet been characterized. Of interest, the major PGN hydrolase in L. monocytogenes is a N-acetylmuramoyl-L-alanyl-amidase that cleaves the bond between the PGN sugar backbone and the peptidic chains. Therefore, L. monocytogenes is unlikely to release significant amounts of muropeptides but rather free peptidic chains and amino sugars (McLaughlan et al. (1998)).

To characterize which signaling pathways are involved in intracellular sensing of Gram-negative bacterial extracts by epithelial cells, it was first demonstrated that this pathway was independent of MyD88, a key adaptor protein of the TLR/IL-1 pathway (Kawai et al. (1999)), since a dominant-negative form of MyD88 was unable to block the activation of the NF-κB pathway induced in digitonin-permeabilized cells by extracts from Gram-negative bacteria, including S. typhimurium AF, S. flexneri and E. coli (FIG. 3c and data not shown). On the contrary, using a dominant-negative form of Nod1 (DN-Nod1), it was possible to efficiently block NF-κB activation induced in digitonin-permeabilized cells by bacterial products from S. flexneri, S. typhimurium and E. coli (FIG. 3d). Several reports have shown that Nod1 activates the NF-κB pathway through the recruitment of Rip2 (Girardin et al. (2001); Inohara, et al., (2000); Chin, et al. (2002); Kobayashi, et al. (2002)). Accordingly, it was observed that a dominant-negative form of Rip2 also blocks the NF-κB pathway induced in digitonin-permeabilized cells by extracts from Gram-negative bacteria (FIG. 3e).

These findings, therefore, demonstrate that Nod1 is the crucial intracellular sensor of bacterial products in epithelial cells and that induction of the Nod1-dependent pro-inflammatory pathway depends upon the ability of a bacterial pathogen, either invasive or extracellular, to translocate Gram-negative PGN to the intracellular environment.

Moreover, additional experiments were conducted on fraction of the GM-dipeptide and surprisingly it has been found that Nod1 is able to sense the tripeptide L-Ala-D-Glu-mesoDAP without the sugar moieties. Therefore, the shortest motif sensed by Nod1, which is identified is the tripeptide.

It will be understood that this invention includes antagonists and agonists of Nod1 that can inhibit or enhance, respectively, one or more of the biological activities of Nod1. Suitable antagonists include small organic or inorganic molecules (i.e., molecules with a molecular weight below about 500), large molecules (i.e., molecules with a molecular weight above about 500), antibodies, and nucleic acid molecules. Agonists of Nod1 also include combinations of small and large molecules.

Thus, the invention features (1) methods for modulating (e.g., decreasing or increasing) an activity of Nod1 by contacting a cell expressing a functional Nod1 with a compound, which activates to Nod1 in a sufficient concentration to modulate the activity of Nod1; and (2) methods of identifying a compound that modulates the activity (e.g., decrease or increase) of Nod1 by contacting the Nod1 with a test compound (e.g., polypeptides, ribonucleic acids, small molecules, large molecules, ribozymes, antisense oligonucleotides, and deoxyribonucleic acids), and detecting and comparing the level of activity of Nod1 in the presence or absence of the test compound.

Compounds that modulate the activity of Nod1 in a cell can be identified by comparing the activity of Nod1 in the presence of a selected compound with the activity of Nod1 in the absence of that compound. A difference in the level of Nod1 activity indicates that the selected compound modulates the expression of Nod1 in the cell.

Exemplary compounds that can be screened in accordance with the invention include, but are not limited to, small organic molecules that are able to gain entry into an appropriate cell and affect the activity of Nod1 protein.

Computer modeling and searching technologies permit identification of compounds, or the improvement of already identified compounds, that can modulate Nod1 activity. Having identified such a compound or composition, the active sites or regions are identified. Such active sites might typically be a binding for a natural modulator of activity. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the modulator (or ligand) is found.

Next, the three dimensional geometric structure of the active site can be determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures can be measured with a complexed modulator (ligand), natural or artificial, which can increase the accuracy of the active site structure determined.

If an incomplete or insufficiently accurate structure is determined, the methods of computer-based numerical modelling can be used to complete the structure or improve its accuracy. Any recognized modelling method can be used, including parameterized models specific to particular biopolymers, such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential Nod1 modulating compounds.

Alternatively, these methods can be used to identify improved modulating compounds from a previously identified modulating compound or ligand. The composition of the known compound can be modified and the structural effects of modification can be determined using the experimental and computer modelling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Examples of molecular modelling systems are the CHARMm and QUANTA programs (Polygen Corporation, Waltham, Mass.). CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modelling of drugs interactive with specific proteins, such as Rotivinen et al., Acta Pharmaceutical Fennica 97:159 [1993]; Ripka, New Scientist 54-57 [Jun. 16, 1988]; McKinaly and Rossmann, Annu Rev Pharmacol Toxicol 29:111 [1989]; Perry and Davies, OSAR: Quantitative Structure-Activity Relationships in Drug Design, pp. 189-193 (Alan R. Liss, Inc. 1989); Lewis and Dean, Proc R Soc Lond 236:125 [1989]; and 141 [1980]; and, with respect to a model receptor for nucleic acid components, Askew et al., J Am Chem Soc 111:1082 [1989]). Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds that could alter binding, one could also screen libraries of known compounds, including natural products or synthetic chemicals, and biologically active materials, including proteins, for compounds that are inhibitors or activators of Nod1 activity.

Compounds identified via assays, such as those described herein, are useful, for example, in elaborating the biological function of Nod1 and for the treatment of disorders associated with aberrant Nod1 activity or expression. Assays for testing the effectiveness of compounds identified with the above-described techniques are discussed below.

In vitro systems may be designed to identify compounds capable of interacting with Nod1 (or a domain of Nod1). Compounds identified may be useful, for example, in modulating the activity of wild type and/or mutant Nod1; may be useful in elaborating the biological function Nod1; may be utilized in screens for identifying compounds that disrupt normal Nod1 interactions; or may in themselves disrupt such interactions.

The principle of the assays used to identify compounds that activate Nod1 involves preparing a reaction mixture of Nod1 (or a domain thereof) and the test compound under conditions and for a time sufficient to allow the two components to interact and activate, thus forming a complex which can be removed and/or detected in the reaction mixture. The Nod1 species used can vary depending upon the goal of the screening assay. In some situations it is preferable to employ a peptide corresponding to a domain of Nod1 fused to a heterologous protein or polypeptide that affords advantages in the assay system (e.g., labeling, isolation of the resulting complex, etc.) can be utilized.

Cell-based assays can be used to identify compounds that interact with Nod1. To this end, cell lines that express Nod1, or cell lines that have been genetically engineered to express Nod1 can be used.

In order to test a compound for inhibitory activity, the reaction mixture is prepared in the presence and absence of the test compound. The test compound can be initially included in the reaction mixture, or can be added at a time subsequent to the introduction of the Nod1 moiety. Control reaction mixtures are incubated without the test compound or with a non-active control compound. The formation of any complexes between the Nod1 moiety and the binding partner is then detected. The formation of a complex in the control reaction, but not in the reaction mixture containing the test compound, indicates that the compound interferes with the interaction of Nod1 and the interactive binding partner. Additionally, complex formation within reaction mixtures containing the test compound and normal Nod1 protein can also be compared to complex formation within reaction mixtures containing the test compound and a mutant Nod1. This comparison may be important in those cases wherein it is desirable to identify compounds that disrupt interactions of mutant but not normal Nod1.

Other methods for identifying compounds capable modulating with Nod1 are disclosed in the Examples.

The invention encompasses methods of diagnosing and treating patients who are suffering from a disorder associated with an abnormal level or rate (undesirably high or undesirably low) of apoptotic cell death, abnormal activity of the Fas/APO-1 receptor complex, abnormal activity of the TNF receptor complex, abnormal activity of a caspase or inflammation of infectious or non-infectious origin by administering a compound that modulates the activity of Nod1. Examples of such compounds include small molecules and large molecules. It will be understood that this invention can be employed to treat a variety of disorders, such as the following. The invention also encompasses TNF receptor complex, abnormal activity of a caspase or inflammation of infectious or non-infectious origin.

Certain disorders are associated with an increased number of surviving cells, which are produced and continue to survive or proliferate when apoptosis is inhibited. These disorders include cancer (particularly follicular lymphomas, carcinomas associated with mutations in p53, and hormone-dependent tumors such as breast cancer, prostate cancer, and ovarian cancer), autoimmune disorders (such as systemic lupus erythematosis, immune-mediated glomerulonephritis), and viral infections (such as those caused by herpesviruses, poxviruses, and adenoviruses).

Failure to remove autoimmune cells that arise during development or that develop as a result of somatic mutation during an immune response can result in autoimmune disease. One of the molecules that plays a critical role in regulating cell death in lymphocytes is the cell surface receptor for Fas.

Populations of cells are often depleted in the event of viral infection, with perhaps the most dramatic example being the cell depletion caused by the human immunodeficiency virus (HIV). Surprisingly, most T cells that die during HIV infections do not appear to be infected with HIV. Although a number of explanations have been proposed, recent evidence suggests that stimulation of the CD4 receptor results in the enhanced susceptibility of uninfected T cells to undergo apoptosis.

A wide variety of neurological diseases are characterized by the gradual loss of specific sets of neurons. Such disorders include Alzheimer's disease, Parkinson's disease, amyotrophic lateral sclerosis (ALS) retinitis pigmentosa, spinal muscular atrophy, and various forms of cerebellar degeneration. The cell loss in these diseases does not induce an inflammatory response, and apoptosis appears to be the mechanism of cell death.

In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses.

Two common disorders associated with cell death are myocardial infarctions and stroke. In both disorders, cells within the central area of ischemia, which is produced in the event of acute loss of blood flow, appear to die rapidly as a result of necrosis. However, outside the central ischemic zone, cells die over a more protracted time period and morphologically appear to die by apoptosis.

Certain inflammatory disorders, both of infectious and non-infectious in origin, could be treated by administering compounds that modulate Nod1 activity. Pathology of inflammatory disorders is associated with inflammation-mediated destruction of the tissue. Inflammatory diseases of non-infectious origin include, but are not limited to, allergy, asthma, psoriasis, rheumatoid arthritis, ankylosing spondylitis, autoimmune diseases (such as systemic lupus erythematosis and glomerulonephritis), and certain cancers. Infectious inflammatory diseases include such infections as those causing gastroenteritis (Shigella spp, Samonella enteritidis, Campylobacter spp., the different strains of diarrheagenic Escherchia coli), gastritis, gastric ulceration, and cancer (Helicobacter pylori), vaginitis (Chlamydia trachomatis,) and respiratory diseases (Pseudomonas aeruginosa, Mycobacteria etc).

Patients who have a disorder mediated by abnormal Nod1 activity can be treated by administration of a compound that alters activity of Nod1. Accordingly, the invention features methods for treating a patient having a disorder associated with the aberrant activity of Nod1 by administering a therapeutically effective amount of a compound (e.g., polypeptide, ribonucleic acid, small molecule, large molecule, ribozyme, antisense oligonucleotide or deoxyribonucleic acid) that decreases or increases the activity of Nod1. Accordingly, the invention features methods for modulating apoptosis by modulating the expression or activity of a gene encoding Nod1.

Agents or modulators, which have a stimulatory or inhibitory effect on Nod1 activity can be administered to individuals for prophylactic or therapeutic treatment of disorders associated with aberrant Nod1 activity. The individual's response to a foreign compound or drug permits the selection of effective agents, and can further be used to determine appropriate dosages and therapeutic regimens. Accordingly, the activity of Nod1 can be determined and used to select an appropriate agent for therapeutic or prophylactic treatment of the individual.

The invention encompasses methods for the detection of peptidoglycan (through MTP) from Gram-negative bacteria in a sample. These methods use the specific interaction between Gram-negative MTP and Nod1 to detect peptidoglycan from Gram-negative bacteria and then Gram-negative bacteria in a sample.

Detection of an interaction between Gram-negative MTP and Nod1 can be done by measuring NK-κB activation in a cell as disclosed in the Examples, for instance.

On the other hand, Nod1 auto-oligomerizes after infection (Inohara et al. (2001)). Based on this characteristic, one can detect Nod1/Gram-negative MTP interaction by the detection of Nod1 oligomerization. For example, detection of Nod1 oligomerization can be performed by means of coupling of Nod1 with a probe. More particularly, the method of the invention can be performed in an acellular system and the oligomerization of Nod1 can be monitored by a physio-chemical reaction. In a particular embodiment, oligomerization of Nod1 can be detected by means of FRET (fluorescence resonance energy transfer) well known by one skilled in the art, which generates a detectable bioluminescent signal.

Nod2 detects either Gram-negative or Gram-positive peptidoglycan (Girardin, et al. (2003); Inohara et al. (2003); Girardin, et al. (2003)). Thus, the methods of the invention can use Nod1 and Nod2 proteins to detect bacterial peptidoglycan, and then the presence of bacteria, in a sample and optionally to determine whether bacteria in said sample are of Gram-negative or Gram-positive origin. More particularly, Nod2 is a general sensor of peptidoglycan from Gram-positive or Gram-negative bacteria through muramyl dipeptide (MDP), while Nod1 is a sensor specific for peptidoglycan from negative bacteria through MTP. Interaction between bacterial peptidoglycan and Nod proteins can be detected by the above-mentioned methods. A method for detecting interaction between MDP and Nod2 is disclosed in (Girardin, et al., (2003)). In a particular embodiment, the method detects bacterial peptidoglycan interaction with Nod proteins by the detection of the oligomerization of Nod1 and Nod2 by means of the FRET technology.

Furthermore, the invention encompasses methods for screening molecules that modulate bacterial peptidoglycan interaction with Nod proteins. In a particular embodiment, said method allows distinguishing molecules that specifically modulate Gram-negative peptidoglycan interaction with Nod proteins, particularly Nod1 protein. The modulation of this interaction is detected by the above-mentioned methods.

### EXAMPLE 1

### Materials and Methods

Bacterial Strains and Products

Bacterial strains used in these studies are the following: S. typhimurium strain C52 and C52-delta flagellin (fliC::aphA-3(Km)fljB5001::Mud(Cm)); E. coli K12; S, flexneri 5a M90T; B. 16 subtilis (from Agnès Fouet, Institut Pasteur); S. aureus (from Olivier Chesneau, Institut Pasteur); L. casei (from Raphaëlle Bourdet-Sicard, Danone Vitapole); L. monocytogenes (Strain EGD, from Pascale Cossart, Institut Pasteur); N. meningitidis LNP8013. Bacterial extracts were prepared from overnight cultures of bacterial strains, diluted to an OD600 of 0.3, sonicated 3 min and filtered (0.2 micron).

Commercial LPS and lipid A were from E. coli O111:B4 (Sigma). Commercial S. aureus PGN was from Fluka Chemicals. Commercial Pam3Cys-Ser-Lys₄-OH lipopeptide was from Roche Diagnostics (Mannheim) and E. coli lipoproteins preparations were provided by Emmanuelle Bouveret and Roland Lioubes (UPR 9027, Marseille).

Pure RE-LPS was from E. coli F515 and purified as previously described (Sanchez Carballo et al. (1999)). Synthetic GM-dipeptide was purchased from Sigma. PGNs of E. coli, S. flexneri and N. meningitidis were purified as described by Glauner et al (Glauner (1988)). PGNs of B. subtilis and S. aureus were purified as described by de Jonge et al (de Jonge et al. (1992)). See also Example 2.

Expression Plasmids and Transient Transfections

The expression plasmid for Flag-tagged Nod1 was from Gabriel Nuñez and has been previously described (Inohara et al., (1999)). The HA-tagged, DN-Nod1 (117-953aa) and myc-tagged “LRR (1-644aa) Nod1 were generated by PCR and cloned into pcDNA3 (Invitrogen) and pRK5 (from Alan Hall, ICRF, London), respectively. DN-MyD88 was from Marta Muzio and the expression plasmid for vsv-tagged DN-Rip2 (7-425aa) was provided by Margot Thome and Jurg Tschopp (University of Lausanne, Switzerland). Transfections were carried out in HEK293 as previously described (Girardin et al. (2001)).

NF-κB Activation Assays

For NF-κB activation assays in digitonin-permeabilized cells, 1×10⁵ HEK293 were grown in 24 well plates and then transfected for 24 h with 75 ng of NF-κB-luciferase reporter gene (IgK luciferase) as previously described (Girardin et al. (2001)). Cells were then incubated for 30 min at 37° C. with 25 μl of sonicated bacterial extracts in 500 μl of permeabilization buffer (50 mM HEPES, pH 7, 100 mM KCl, 3 mM MgCl2, 0.1 mM DTT, 85 mM sucrose, 0.2% BSA, 1 mM ATP and 0.1 mM GTP) with or without 10 μg/ml digitonin (Sigma). Permeabilization buffer was then removed and replaced with medium (DMEM, Gibco) plus 10% fetal-calf serum (Gibco) for 4 hours before processing for luciferase measurements as described previously (Girardin et al. (2001)). For dominant-negative studies presented in FIG. 3, increasing amounts of dominant-negative constructs were co-transfected with the NF-κB reporter plasmid. Experiments were then performed as detailed above.

Studies examining the activation of NF-κB by LPS, lipid A, lipoproteins or the purified PGNs in cells over-expressing Nod1 were carried out as described by Inohara et al. (Inohara et al. (2001)). Briefly, HEK293 cells were transfected overnight with 10 ng of Nod1. At the same time, the LPS, lipoproteins or peptidoglycan preparations were added and the NF-κB-dependent luciferase activation was then measured following 24 h of co-incubation. It is presumed that the transfection reagent plus the added DNA aids in the uptake of bacterial products into the cells since extracellular addition of these products to cells previously transfected and washed to remove the liposome reagent does not lead to NF-κB activation (data not shown).

NF-κB-dependent luciferase assays were performed in duplicate and data represent at least 3 independent experiments. Data show mean±SEM and are expressed as fold activation compared to vector expressing cells or as fold synergy compared to the level of NF-κB activation of Nod1-expressing cells (for 10 ng of Nod1, NF-κB activation is approximately 5 fold compared to vector-expressing cells).

For immunofluorescence studies, NF-κB activation was assessed by nuclear translocation of NF-κB p65 in HeLa, Caco-2 or isolated intestinal epithelial cells following microinjection of bacterial products (diluted 1:1 with FITC-dextran) as previously described (Philpott et al., (2000)). At least 50 microinjected cells were examined per coverslip and experiments were performed at least 2 times independently with similar results.

Interleukin-8 Production

To measure Nod1-dependent IL-8 produced in epithelial cells by muropeptides, 5×10⁵ HeLa cells were seeded into each well of a twelve well plate and transfected the following day with 10 ng Nod1 plus the individual muropeptides (as described above) or treated with IL-1 as a positive control. Eighteen hours later, supernatants were collected and assayed for IL-8 as previously described (Philpott et al. (2000)) using an ELISA kit (R and D Systems).

Western Blot and Immunoprecipitations

Western blot and immunoprecipitations were carried out as previously described (Girardin et al. (2001)). The Nod1 polyclonal antibody was obtained by immunization of rabbits with two peptides corresponding to aa 1-15 and 567-582 of Nod1. Serum was collected, affinity purified and verified to be specific for Nod1. The Nod2 polyclonal antibody was from Cayman Chemical (Ann Arbor, Mich.).

### EXAMPLE 2

### Preparation of Highly Purified PGNs from Gram-Negative and Gram-Positive Bacteria

Bacterial strains used to prepare PGN are the following: E. coli K12; S, flexneri 5a M90T (wildtype); N. meningitidis; B. subtilis 168; S. aureus COL (from Olivier Chesneau, Institut Pasteur). PGNs of E. coli and S. flexneri were purified as described by Glauner et al (Glauner (1988)). PGNs of B. subtilis and S. aureus were purified as described by de Jonge et al (de Jonge, et al. (1992)). Briefly, bacteria were harvested in exponential growth phase at an optical density (600 nm) of 0.4-0.6 and quickly chilled in an ice-ethanol bath to minimize PGN hydrolysis by endogeneous autolysins. Pellets were resuspended in ice-cold water and added drop by drop to 8% boiling SDS. Samples were boiled for 30 minutes allowing immediate inactivation of autolysins. Polymeric PGN, which is insoluble, was recovered by centrifugation and washed several times until no SDS could be detected. SDS assay was done as described by Hayashi (Hayashi (1975)). SDS treatment removes contaminating proteins, non-covalently bound lipoproteins and LPS. Gram-positive bacterial samples were physically broken with acid washed glass beads (<100 nm). The PGN fraction was recovered by differential centrifugation to remove cellular debris. All PGNs were further treated with α-amylase to remove any glycogen and with trypsin (3× crystallized trypsin, Worthington) digestions to remove covalently bound proteins (LPXTG proteins in Grampositive bacteria) or lipoproteins (Gram-negative bacteria). Samples were further boiled in 1% SDS to inactivate trypsin and were washed to remove SDS. Gram-positive bacterial samples were treated with 49% hydrofluoridic acid during 48 hours at 4° C. This mild acid hydrolysis allows removal of secondary polysaccharides covalently bound to the PGN by phosphodiester bonds such as teichoic acid, capsules, poly-(β,1-6 GlcNAc), etc. Further treatment of both Grampositive and Gram-negative PGNs included washes with 8 M LiCl, 0.1 M EDTA to remove any polypeptidic contaminations and with acetone to remove lipoteichoic acids or any traces of LPS. Samples were lyophilized to measure PGN amounts. Purity of samples was assessed by HPLC amino acid and saccharide analysis after HCl hydrolysis (see also FIG. 4).

### EXAMPLE 3

### Analysis of N. meningitidis PGN by Reverse-Phase HPLC and Mass Spectroscopy

Peptidoglycans of N. meningitidis or S. flexneri were digested by the muramidase mutanolysin (M1, Sigma) to generate the entire spectrum of muropeptides for both species. The muropeptides were reduced with sodium borohydride and separated by reverse-phase HPLC as described by Glauner (Glauner (1988)). Individual muropeptide peaks were collected and directly used for biological assays. For mass spectrometry analysis, the different muropeptide fractions from N. meningitidis peptidoglycan were further desalted by HPLC as described by Garcia-Bustos and Dougherty (Garcia-Bustos et al. (1987)). Desalted muropeptides were analyzed by MALDI-TOF as described by Xu et al. (Xu et al. (1997)).

These molecular masses were found for the following analyzed fractions: fraction 3 [M+H]⁺871, 6214 m/z!; [M+Na]⁺: 893, 3633 m/z; [M+2Na−H]+ 915, 3518 m/z which is consistent with the GlcNAc-MurNAc-L-Ala-D-Glu-mesoDAP structure (calculated mass 870); fraction 6 [M+H]+942, 4512 m/z; [M+Na]+ 964, 4689 m/z [M+2Na−H]⁺ 986, 4429 m/z; [M+3Na−2H]⁺ 1008, 4321 m/z, which is consistent with a GlcNAc-MurNAc-L-Ala-D-Glu-mesoDAP-DAI structure (calculated mass 941); fraction 17 a mixture of two muropeptide species: 1) [M+H]⁺ 851, 3460 m/z; [M+Na]⁺ 873, 3422 m/z, [M+2Na−H]⁺ 895, 3219 m/z; [M+3Na−2H]⁺ 917, 3115 m/z which is consistent with a GlcNAc-anhydro-MurNAc-L-Ala-D-GlumesoDAP structure (calculated mass 850) and 2) [M+H]⁺ 1865, 5588 m/z [M+Na]⁺ 1887, 5331 m/z, [M+2Na−H]⁺ 1909, 5753 m/z; [M+3Na−2H]⁺ 1931, 5625 m/z which is consistent with the muropeptide dimer GlcNAc-MurNAc-L-Ala-D-Glu-mesoDAP(GlcNAc-MurNAc-L-Ala-D-Glu-mesoDAP-D-Ala)-D-Ala structure (calculated mass 1864).

### EXAMPLE 4

### Nod1 Pathway

The Nod1 pathway is stimulated by commercial LPS and Gram-negative bacterial PGNs. FIG. 1a shows the stimulation of the Nod1 pathway by commercial E. coli LPS but not protein-free pure LPS or lipid A. FIG. 1b shows that lipopeptide or E. coli lipoproteins (lipidated and de-lipidated, i.e., “Lpp” and “soluble E.c Lpp”) fail to activate the Nod1 pathway. FIG. 1c shows that Nod1 mediates NF-κB responsiveness to Gram-negative PGNs (E. coli, S. flexneri, N. meningitidis), but not Gram-positive PGNs (B. subtilis, S. aureus or commercial S. aureus PGN). No synergistic activation of NF-κB by E. coli PGN was observed in cells expressing ΔLRR-Nod1 as shown in FIG. 1d. LPS, lipid A, lipoproteins preparations were used at 10 μg/ml. PGN preparations were used at 1 μg/ml.

### EXAMPLE 5

### Characterization of the PGN Motif Detected by Nod1

The characterization of the PGN motif detected by Nod1 is shown in FIG. 2. In FIG. 2a, PGN muropeptides from N. meningitidis were separated by reverse-phase HPLC and subsequently analysed by mass spectroscopy (see supporting on-line text). N.D.(a), not determined for this particular HPLC fractionation, but found to be O-acetylated dimeric GM-tetrapeptide in subsequent HPLC fractionation and mass spectroscopy analyses. This fraction was also negative for Nod1 stimulation in additional experiments (data not shown). N.D.(b), fraction unknown. In FIG. 2b, 1% of the volume of each individual HPLC fraction was tested for stimulation of the Nod1 pathway showing that fraction 3, corresponding to GM-tripeptide, activates Nod1. Fraction 17, corresponding to a mixture of dimeric GM-tetrapeptide (does not activate Nod1) and anhydrous GM-tripeptide, activates Nod1. FIG. 2c is a schematic representation of N. meningitidis PGN detailing the Nod1-active motif from fraction 3 (GM-tripeptide) as determined by mass spectroscopy. In FIG. 2d, equivalent amounts (10 ng) of GM-dipeptide (GM-di; synthetic source), GM-tripeptide (GMtri), or GM-tetrapeptide (GM-tetra) were tested for stimulation of Nod1-, Nod2- or TLR2-dependent activation of NF-κB. FIG. 2e relates to synergistic activation of Nod1 or Nod2 by N. meningitides and B. subtilis PGNs. FIG. 2f shows the production of the pro-inflammatory chemokine, IL-8, in HeLa epithelial cells stimulated with the GM-tripeptide in the presence of Nod1 but not the GM-dipeptide or tetrapeptide. IL-1 (10 ng/ml) stimulation of IL-8 production is shown as a positive control. Buff, buffer diluent of the muropeptides.

### EXAMPLE 6

### Intracellular Detection of Gram-Negative Bacteria

Intracellular detection of Gram-negative but not Gram-positive bacterial products in epithelial cells through Nod1/Rip2 but not MyD88 is shown in FIG. 3.

Extracts from Gram-negative (S.t, Salmonella typhimurium; S.t ΔF, S. typhimurium-delta flagellin; E.c, Escherichia coli; S.f, Shigella flexneri) and Gram-positive (B.s, Bacillus subtilis; S.a, Staphylococcus aureus; L.c, Lactobacillus casei; L.m, Listeria monocytogenes) bacteria were added to HEK293 cells permeabilized or not by digitonin (10 μg/ml) and NF-κB activity was measured after 4 h using an NF-κB-luciferase reporter assay. The results are shown in FIG. 3a.

HeLa cells were microinjected with either Dextran-FITC only (buffer) or with bacterial extracts and stained for NF-κB p65. 100% of cell microinjected with Gram-negative bacterial supernatants show translocated in FIG. 3b, NF-κB whereas no active cells were observed with the Gram-positive bacterial supernatants. DAPI stain shows position of nuclei. Arrows point to translocated NF-κB p65 in the nuclei of activated cells.

No effect of dominant-negative MyD88 (DN-MyD88; 0, 20, 50 ng) on Gram-negative bacterial extracts-induced NF-κB activity is shown in FIG. 3c, yet inhibition of IL-1-induced NF-κB activation (IL-1, 10 ng/ml).

Inhibition of Gram-negative bacterial extracts-induced NF-κB activity in digitonin-permeabilized HEK293 cells transfected with Nod1 117-953aa (DN-Nod1; 0, 200, 400 ng) as shown in FIG. 3d.

Inhibition of Gram-negative bacterial extracts-induced NF-κB activity by dominant-negative Rip2 (DN-Rip2; 0, 50, 10 ng) as shown in FIG. 3e.

### EXAMPLE 7

### Response of Intestinal Epithelial Cells from Mice Deficient in Nod1

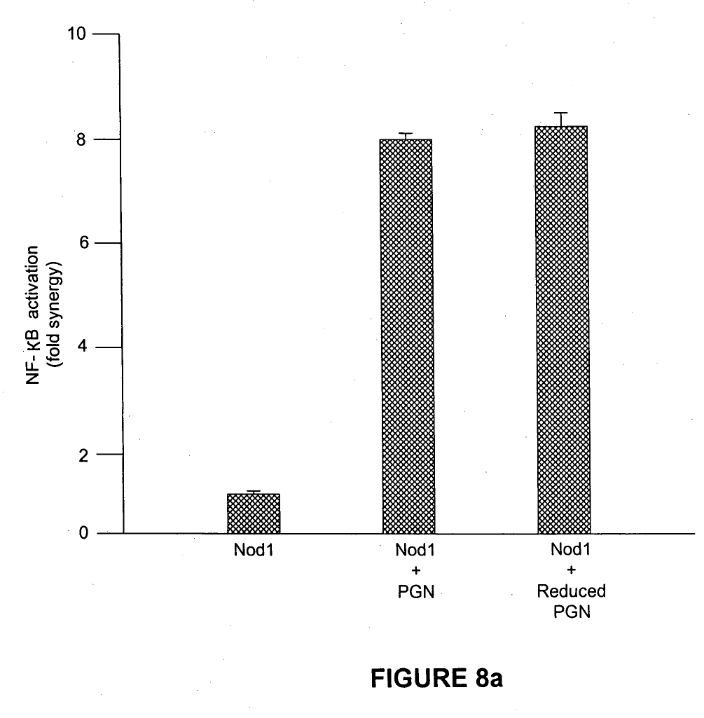

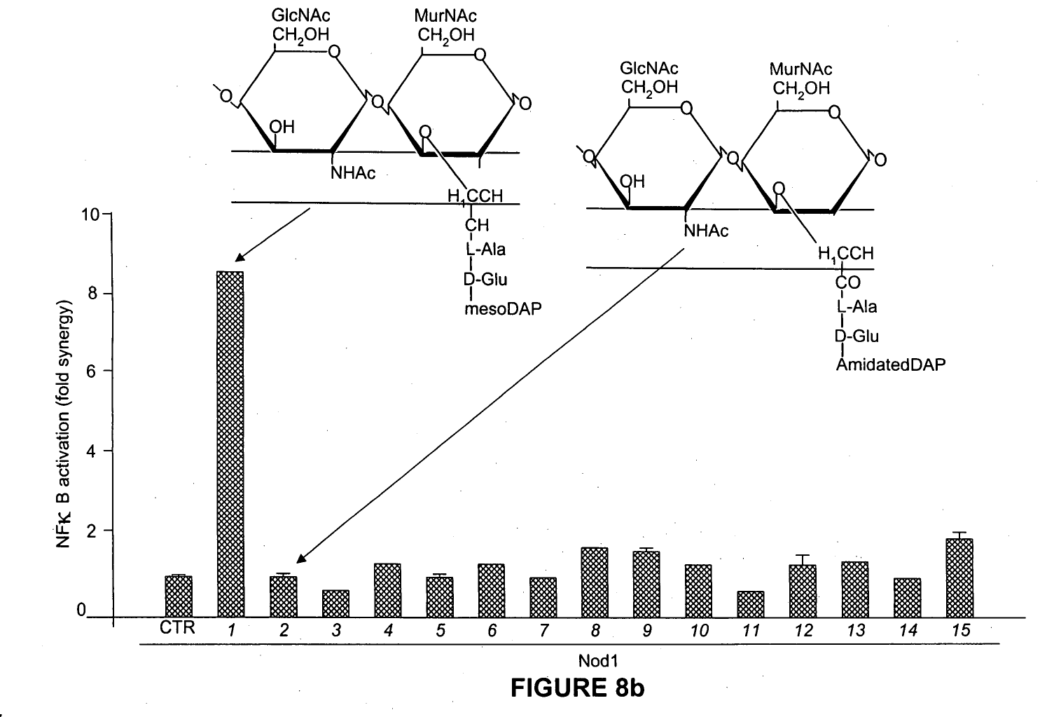

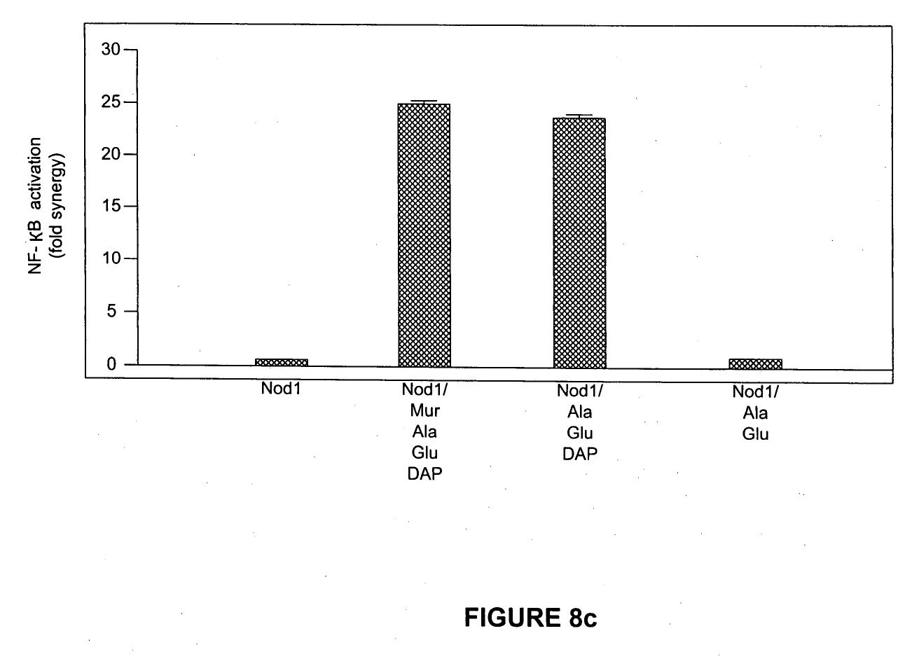

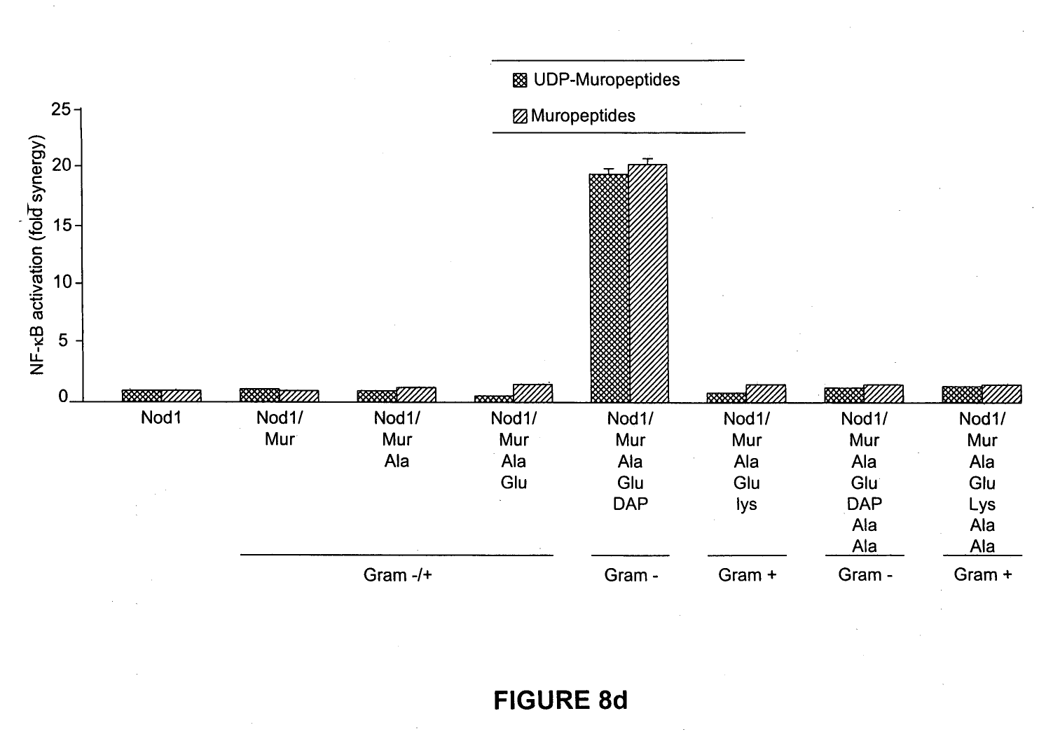

Intestinal epithelial cells from mice deficient in Nod1 do not respond to bacterial supernatants as shown in FIG. 8. Microinjection of Gram-negative but not Gram-positive bacterial supernatants activates NF-κB in isolated intestinal epithelial cells from wild-type mice, as observed by nuclear translocation of the p65 subunit of NF-κB (as described in FIG. 3b). In contrast, cells from Nod1-deficient mice are not activated by bacterial supernatants, although TNFα (10 ng/ml) can efficiently stimulate NF-κB nuclear translocation in these cells. 98% of wild-type cells microinjected with Gram-negative bacterial supernatants showed translocated NF-κB in the nucleus whereas no active cells were observed with Gram-positive bacterial supernatant microinjection. In the case of the Nod1-deficient cells, no active cells were observed in either case. Extracellular addition of either Gram-negative or Gram-positive bacterial supernatants failed to stimulate NF-κB nuclear translocation in wild-type or Nod1-deficient cells (data not shown).

In Drosophila, the Toll pathway detects both Gram-positive bacteria and fungi, while the lmd pathway is specific to Gram-negative bacterial sensing (Lemaitre et al. (1996) 10). Recently, two peptidoglycan-recognition proteins (PGRPs) have been shown to play a key role in the discriminatory detection of bacteria in Drosophila (Michel et al., (2001); Choe et al. (2002); Gottar, et al. (2002); Ramet et al., (2002)). PGRP-SA is involved in Gram-positive bacterial recognition in the Toll pathway while PGRP-LC acts upstream of lmd in Gram-negative bacterial sensing. However, definitive proof that this discriminatory detection actually relies on PGN is still lacking.

This invention has shown that in mammalian cells, Nod1-dependent detection of bacteria relies on the sensing of a Gram-negative PGN motif. Indeed, this invention demonstrates that GM-tripeptide and GM-dipeptide form a new class of bacterial PAMPs, which are recognized differentially by Nod1 and Nod2, respectively. These PGN motifs are naturally occurring degradation products released from the bacteria during growth. Therefore, the peptidic composition of the PGN degradation products either released by the bacteria or processed by the host cell in the lysosomal compartment is critical in defining the host response towards bacterial infection. In this respect, the characterization of the PGN motifs sensed by Nod1 and Nod2 suggest that these two molecules have complementary and non-overlapping functions that contribute to innate immunity. Moreover, the results of this invention have demonstrated that Nod1 is likely the sole sentinel molecule in the epithelial barrier allowing intracellular detection of bacteria through PGN sensing, thereby highlighting its key role in innate immune defense.

### EXAMPLE 8

### Cell Culture

Bacterial strains were routinely subcultured on blood agar medium (Blood Agar Base No. 2 {Oxoid}), supplemented with 10% horse blood (bioMerieux) 37. Broth cultures were prepared in 10 ml Brain Heart Infusion broth (BHI; Oxoid) containing 10% heat-inactivated fetal calf serum (FCS; Invitrogen Life Technologies). Cultures were shaken at 140 rpm in tissue culture flasks (Falcon), and incubated under microaerobic conditions, at 37° C. Solid and liquid culture media were supplemented with kanamycin (20 μg/ml) or chloramphenicol (10 μg/ml), as appropriate. Viable counts of H. pylori bacteria were performed by serial dilution of culture suspensions in sterile peptone trypsin broth (Ferrero et al./(1998)).

The cagA mutant was constructed by natural transformation in H. pylori 251 as described previously (Chevalier et al. (1999)). cagM gene inactivation was performed using a construct from mini-Tn3-Km mutagenesis of a library of cloned H. pylori genomic DNA (Jenks, et al. (2001)). The transposon insertion site was mapped to nucleotide position no. 935 in the cagM gene of H. pylori 26695. H. pylori slt and lysA single mutants were generated from recombinant plasmids in which the corresponding genes had been disrupted by a non-polar kanamycin resistance marker (Skouloubris et al. (1998)). The lysAcagM double mutant was constructed by cagM gene disruption using a non-polar version of the chloramphenicol resistance catGC cassette, described by Heuermann et al (Heuermann et al. (1998)). All H. pylori mutants were verified by PCR and/or DNA sequencing, using standard molecular biology techniques.

Highly purified H. pylori LPS was prepared by hot phenol-water extraction and subsequent enzymatic treatments with DNase, RNase and proteinase K (Sigma Chemical Co.) and by ultracentrifugation, as described previously (Moran et al. (1992)). H. pylori PG was purified from H. pylori bacteria using a modified version of the technique of Glauner et al. (Girardin et al., (2003); Glauner (1988)). Briefly, bacteria were harvested in exponential growth phase (A₆₀₀ 0.6-0.8) and quickly cooled in an ice-ethanol bath. PG was extracted by boiling bacteria in an equal volume of 8% (w/v) SDS. After differential centrifugation, PG-containing fractions were washed several times to remove SDS. The fractions underwent enzymatic treatments to remove traces of DNA, RNA or proteins, and were further boiled in 1% SDS. SDS was removed by several wash procedures. PG samples were sequentially incubated in 8 M LiCl, centrifuged, incubated with 0.5 M EDTA, and washed. Acetone extraction of lipid-containing contaminants was performed by ultrasonic sonication. Finally, PG samples were centrifuged, washed twice and stored at −20° C. until used. Purified H. pylori PG had no activity on TLR2- or TLR4-transfected HEK293 cells (unpublished data, SEG and IB). Muropeptides from H. pylori 26695 were purified by HPLC on muramidase-digested peptidoglycan, as described elsewhere (Girardin et al. (2003). Individual muropeptide peaks were collected and directly used in NF-κB reporter assays in HEK293 cells. The muropeptides present in each fraction were identified by MALDI-TOF analysis of the fractions following desalting by HPLC (Girardin et al. (2003).

HEK293, HeLa and AGS cell lines were grown routinely in Dulbecco's Minimal Essential Media (MEM), Eagle's MEM or RPMI 1640, respectively, containing 10% FCS, and supplemented with 100 IU ml⁻¹ penicillin, 100 mg I⁻¹ streptomycin, and 10 mM l-glutamate (all reagents from Invitrogen), at 37° C. in 5% CO₂. HEK cells were not serum-starved prior to co-culture experiments. AGS cells that had been serum-starved overnight were washed three times and the RPMI 1640 medium replaced with antibiotic-free, serum-free medium. Overnight (16-20 h) liquid cultures of Helicobacter strains were harvested by centrifugation at 4000 g for 15 min (at 4° C.) and washed twice in phosphate-buffered saline (PBS, pH 7.4) prior to resuspension in the appropriate cell culture medium. Helicobacter bacteria were added to cell cultures at an MOI of 10-100. Viable counts of the bacterial suspensions were determined by serial plating.

The expression of nod1 mRNA was detected by RT-PCR in HEK293, HeLa and AGS cells. For this, total RNA was prepared from cells using TRIzol reagent (Invitrogen). Purified RNA (2 μg) was reverse transcribed with Superscript II RNase H (Invitrogen) according to the manufacturer's instructions. PCR was then performed using forward (CCTGACAAGGTCCGCAAA) [SEQ ID NO: 3] and reverse (GTCCATGTAGATCTCCTCCA) [SEQ ID NO: 4] oligonucleotides specific for human nod1. The quantity of cDNA in samples was standardized using β-actin-specific forward (GGGTCAGAAGGATTCCTATG) [SEQ ID NO: 5] and reverse oligonucleotides (GGTCTCAAACATGATCTGGG) [SEQ ID NO: 6]. cDNA samples were subjected to one cycle of heat denaturation at 95° C., for 3 min, followed by 40 PCR cycles, each comprising successive incubations at 95° C., 60° C. and 72° C., for 20 sec each. A further extension step was performed at 72° C. for 7 min. The respective amplification products were identified by ethidium bromide staining of agarose gels.

IL-8 production by AGS cells was determined from culture supernatants (6 and 24 h post-stimulation) using a double sandwich ELISA technique (R&D Systems).

### EXAMPLE 9

### Cell Transfection and siRNA Assays

HEK293 cells were plated in 24 well plates at a density of 1×10⁵ cells and transfected the following day with Igκ-luciferase reporter DNA, using FuGene6 reagent medium (Girardin et al. (2001)). For dominant-negative studies, cells were co-transfected with ΔCARDNod1 DNA (Bertin et al. (1999)). The transfected cells were incubated overnight and co-cultured for 4 h with live bacteria, prior to lysis of the cells (Girardin et al. (2001)). The activities of highly purified H. pylori LPS and PG on NF-κB activation was studied in HEK293 cells over-expressing Nod1, as described previously (Girardin et al. (2003)). Briefly, cells were first co-transfected with Igκ-luc reporter construct, Nod1 DNA (10 ng; Bertin et al. (1999)), and purified H. pylori LPS (10 μg) or peptidoglycan (1 μg). Luciferase activities were determined in cells following 24 h of co-incubation with the bacterial products. AGS cells were plated in 24 well plates at a density of 8×10⁴ cells and transfected the following day using FuGene6 reagent (Roche Diagnostics) with Igκ-luciferase and β-galactosidase reporter (Invitrogen) constructs (Philpoft et al. (2000). Thirty-six hours post-transfection, cells were infected with bacteria, as described above. Six hours later, lyzed cells were assayed for luciferase and β-galactosidase activities using a Berthold 96-well luminometer.

siRNA of H. pylori-induced NF-κB reporter activity was performed using the technique described by InvivoGen. For this, HEK293 cells that had been plated at a density of 1.0×10⁴ cells in FCS-enriched DMEM were transfected 1 day later with varying concentrations of the Nod1siRNA construct together with pCDNA3 (total DNA concentration, 500 ng) using FuGene6 reagent. The Nod1siRNA construct contained a sequence (5′-ACAACTTGCTGAAGAATGACT-3′) [SEQ ID NO: 1] which is specific for the CARD in human nod1. A sequence (5′-GCAAGCTGACCCTGAAGTTCA-3′) [SEQ ID NO: 2] with no homology to human genes (EGFPsiRNA; InvivoGen) was used as a control. For Western blot studies, the cells were co-transfected with 20 ng of Nod1 reporter construct as well as varying concentrations of the respective siRNA constructs, prior to lysis at 48 h post-transfection. The cell extracts were analyzed using standard SDS-PAGE and Western blotting techniques. Nod1 production in samples was determined using an ‘in-house’ rabbit anti-Nod1 antibody (diluted 1:2000) and chemiluminescence detection reagent (picoECL, Amersham Biosciences). β-tubulin detection was performed on “stripped” membranes using a mouse monoclonal antibody (1:10,000; Sigma). For luciferase reporter assays, the cells that had been transfected with one of the siRNA constructs were transfected 2 days later with Igκ-luciferase plasmid, incubated for 24 h, then co-cultured for 7 h with H. pylori bacteria, prior to analysis.

### EXAMPLE 10

### Peptidoglycan Translocation Studies

AGS cells were plated at a density of 0.3×10⁴ cells in 8-well Labtek (Costar) glass slides and cultured overnight. Specific ³H-radiolabelling of H. pylori PG was performed by cultivation (6-8 h) of H. pylori 26695 lysA or lysAcagM mutant bacteria in FCS-enriched BHI medium supplemented with 20 μM ³H-labeled mDAP. The bacteria were centrifuged at 3000×g (10 min, 4° C.), and resuspended in cell culture medium. The efficiency of PG tritiation was similar for lysA or lysAcagM mutants (CC, IGB, unpublished data). AGS cells were co-cultured for 3-4 h or 16 h with radio-labeled bacteria, at an MOI of 10-100. As negative controls, AGS cells were either left untreated or were co-cultured with heat-killed (60° C., 20 min) H. pylori lysA bacteria. The AGS cells were washed 3 times with PBS, prior to fixation (20 min) in 3% (v/v) paraformaldehyde in PBS. To prepare slides for β-imager analysis, cells were washed once in PBS, dehydrated in successive washes of 90% and 100% ethanol, then air-dried. Quantitative determination of the radioactivity in AGS cells was determined using a Micro-Imager (Biospace, Paris, France) (Laniece et al. (1998)). The presence of tritiated PG was detected by the immersion of the fixed slides in nuclear emulsion (Kodak NTB-2) Rougeot et al. (1997)). The slides were processed as described previously Rougeot et al. (1997)), counter-stained with Giemsa, and analyzed by the Micro-Imager machine. Immunohistochemistry was performed on slides using anti-H. pylori whole cell rabbit (diluted 1:600), prior to their immersion in nuclear emulsion.

### EXAMPLE 11

### Mouse Colonization

Nod1^{-/-} and Nod1^{+/+} mice on a C57BL/6×SV129 background were generated under specific pathogen-free conditions in the animal facilities of the Institut Pasteur (Paris), from breeding pairs that had been kindly supplied by J. Bertin (Millenium Inc, MA). Six to 8 wk-old female and male mice were used throughout. These mice were shown to be Helicobacter-free by bacteriological and serological assays (data not shown). Animals were housed in polycarbonate cages in isolators and fed a commercial pellet diet with water ad libitum. Animal handling and experimentation was performed in accordance with institutional guidelines and current French legislation (Law No. 87-848).

Nod1^{-/-} and wild-type mice were inoculated intragastrically with H. pylori or H. felis isolates (Ferrero et al. (1998)). H. pylori infection was determined by quantitative culture of gastric tissue fragments from mice sacrificed at the appropriate times (Ferrero et al. (1998)). Since H. felis does not reproducibly form isolated colonies on culture plates, H. felis colonization was assessed by histological analyses performed on multiple Giemsa-stained sections of paraffin-embedded tissues (Ferrero et al., (1995)).

Primary gastric epithelial cells. Four week-old female and male mice were used for the derivation of primary gastric epithelial cell cultures, as described previously (Ahman et al. (2002)). Briefly, the stomachs were removed and put into Hanks buffered salts solution (Invitrogen) containing 0.2% bovine serum albumin (BSA). The stomachs were opened and cut into <0.5 mm fragments, prior to centrifugation at 800 rpm for 3 min. The pelleted material was digested successive times with enzyme solution (300 U/ml collagenase in HBSS+BSA, pH7.4), by incubation at 37° C. for 1 h, with shaking. The resulting cell pellets were resuspended in DMEM supplemented with 20% FCS, l-glutamine, penicillin and streptomycin. Aliquots (500 μl) of these suspensions were added together with 1.5 ml of supplemented DMEM to 6-well tissue culture plates. The cells were grown at 37° C. in 5% CO₂, for 72 h. Bacterial co-culture experiments (MOI of 10-100) were performed with serum-starved cells. The cultured cells were analyzed by immunofluorescence as described previously (Widmer et al. (1993)).

### EXAMPLE 12

### H. pylori Induces NF-κB Activation in HEK293 Cells Via a caqPAI-Dependent Mechanism

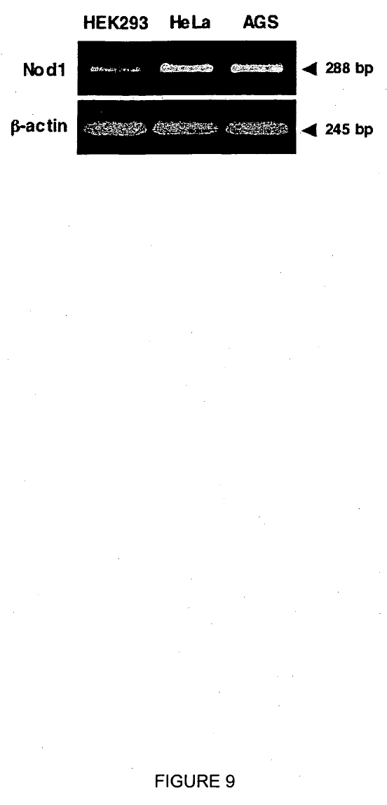

Epithelial cell signaling in response to H. pylori was investigated by measuring NF-κB activation in HEK293 cells, using an NF-κB-dependent luciferase reporter gene assay. This cell line has previously been shown to respond to intracellular stimulation with Gram-negative whole bacteria (Girardin et al. (2001)) or PG (Girardin et al. (2003)), via a Nod1-dependent pathway. Indeed, nod1 expression was detected in this cell line, as well as in AGS gastric epithelial cells (FIG. 9). HEK293 cells do not produce functional forms of TLR2 and TLR4 (Maeda et al. (2001); Girardin et al. (2003)), and are therefore non-responsive to Gram-negative lipoproteins and LPS, respectively.

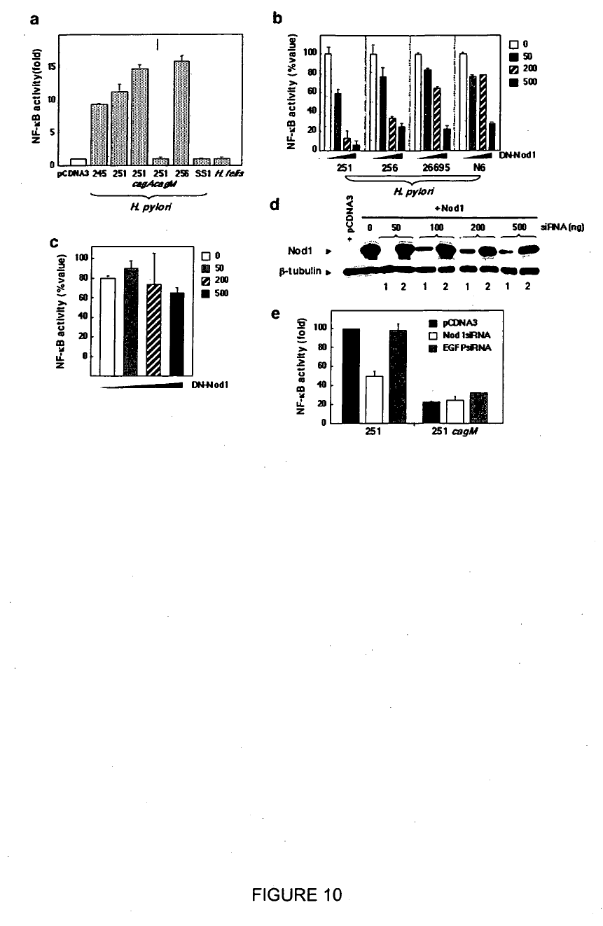

Several H. pylori clinical and laboratory strains were identified as being able to induce NF-κB-dependent luciferase expression in HEK293 cells (FIG. 10a). A perfect correlation was found between the data from NF-κB reporter studies in HEK293 cells, and those from NF-κB activation and/or IL-8 secretion assays in the AGS gastric epithelial cell line (Table 1). Accordingly, gastric Helicobacter isolates in which the cagPAI was either absent (Helicobacter felis) or, alternatively, non-functional (H. pylori SS1; (Crabtree et al. (2002)), did not induce NF-κB-luciferase activity in HEK293 cells (FIG. 10a). As previously shown for AGS cells, genetic disruption of the cagM gene, whose product is essential for type IV secretion system functions (Fischer et al. (2001); Selbach (2002)), abrogated H. pylori induction of NF-κB-luciferase activity in HEK293 cells (FIG. 10a). Conversely, a cagA mutant, which retains its pro-inflammatory effects on AGS cells (Fischer et al. (2001); Selbach et al. (2002)), was unaffected in its ability to induce NF-κB activation in these cells (FIG. 10a). Collectively, these data show that H. pylori-mediated NF-κB activation in HEK293 cells was strictly dependent on the presence of a functional cagPAI.

### EXAMPLE 13

### Nod1 Mediates H. Pylori-Induced NF-κB Activation in Epithelial Cells

To investigate the putative role of a Nod1-dependent signaling pathway in epithelial cell sensing of cagPAI-positive H. pylori, HEK293 cells were co-transfected with the NF-κB-luciferase reporter construct and a dominant negative-Nod1 plasmid (DN-Nod1), in which the caspase-recruitment domain (CARD) necessary for NF-κB activation (Bertin et al. (1999); Inohara et al. (1999)) had been deleted. The addition of increasing concentrations of DN-Nod1 abrogated the effect of cagPAI-positive H. pylori strains on NF-κB activation by up to 90%, when compared to cells receiving vector DNA alone (FIG. 10b). This effect was observed for 4 cagPAI-positive isolates (H. pylori strains 251, 256, 26695, and N6), as well as for a cagA mutant (FIG. 10b, and data not shown). Conversely, transfection with the DN-Nod1 construct at these doses did not abrogate TNF-induced NF-κB reporter activity in HEK293 cells, as described previously (FIG. 10c) (Girardin et al. (2001)).

To further confirm the role of the Nod 1 pathway in H. pylori recognition, siRNA experiments were performed with a plasmid construct (Nod1siRNA) containing a cloned sequence homologous to a 21-nucleotide region within the CARD of Nod1. Differences in Nod1 synthesis and activity were determined in HEK293 cells using Western blotting and NF-κB reporter assays, respectively. Since endogenous Nod1 is below the level of detection by Western blotting (FIG. 10d; pCDNA3-transfected cells), HEK293 cells were first transfected with wild-type Nod1 and the effects of the siRNA constructs assessed on over-expressed levels of the molecule. In these conditions, it was observed that co-transfection of the cells with Nod1siRNA inhibited the synthesis of exogenously expressed Nod1 in HEK293 cells (FIG. 10d). In contrast, an siRNA construct with no homology to human genes (EGFPsiRNA) did not affect Nod1 synthesis (FIG. 10d). HEK293 cells that were transfected with the Nod1siRNA construct prior to stimulation with H. pylori, also exhibited decreased NF-κB reporter activities of up to 50%, when compared to cells transfected with a control plasmid (pCDNA3) alone. No significant decreases in NF-κB reporter activity were observed in Nod1siRNA-transfected HEK293 cells after stimulation with an H. pylori cagM mutant, nor in EGFPsiRNA-transfected cells stimulated with wild-type H. pylori bacteria (FIG. 10d). It is noteworthy that a longer time-period (72 h) was required to alter Nod1-dependent NF-κB activity, than that required to affect exogenous Nod1 synthesis (48 h). This is probably due to the relatively long half-life of endogenous Nod1, which was able to continue driving NF-κB reporter activity, even after nod1 transcription had been effectively inhibited by Nod1siRNA.

Taken together, the preceding data demonstrated that H. pylori-induced NF-κB activation in epithelial cells was: 1) dependent on the presence in the bacteria of a functional type IV secretion system, and 2) mediated by intracytoplasmic signaling via Nod1. Thus, it is hypothesized that Nod1 might respond to an effector molecule that is delivered to the cytoplasm of host epithelial cells by the cagPAI type IV secretion system.

### EXAMPLE 14

### Nod1 Recognizes a Specific Motif of H. pylori PG

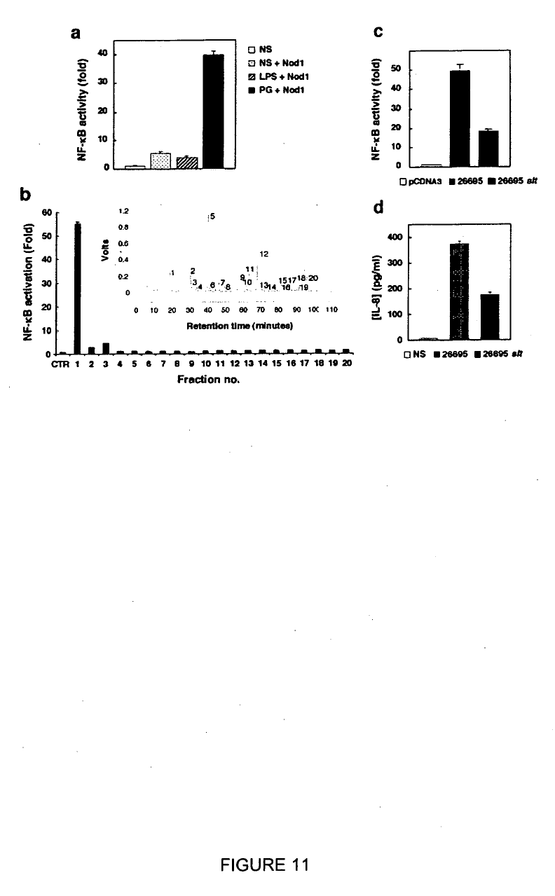

The PG of Gram-negative cell walls was recently identified as the microbial product that is recognized by Nod1 (Girardin et al. (2003). In agreement with this finding, intracytoplasmic presentation of H. pylori PG, together with DNA encoding exogenous Nod1, induced high levels of NF-κB activation in HEK293 cells (FIG. 11a; P=0.03). Under identical experimental conditions, purified H. pylori LPS was a poor stimulus of Nod1-dependent induction of NF-κB (FIG. 11a). The specific motif of H. pylori PG that was recognized by Nod1 was identified by testing different muropeptide fractions from H. pylori 26695 for their effects on NF-κB reporter activity in HEK293 cells. Reversed-phase high-performance liquid chromatography (HPLC) of muramidase-digested PG generated a single fraction (FIG. 11b, inset; number 1) with very high NF-κB-inducing activity for these cells (FIG. 11b). Mass spectroscopy of this fraction revealed a mass/charge ratio m/z of 893, which was consistent with a meso diaminopimelate (mDAP)-containing N-acetylglucosamine-N-acetylmuramic acid (GM-tripeptide) structure. This structure corresponds to the PG motif that is specifically recognized by Nod1 (Girardin et al. (2003)).

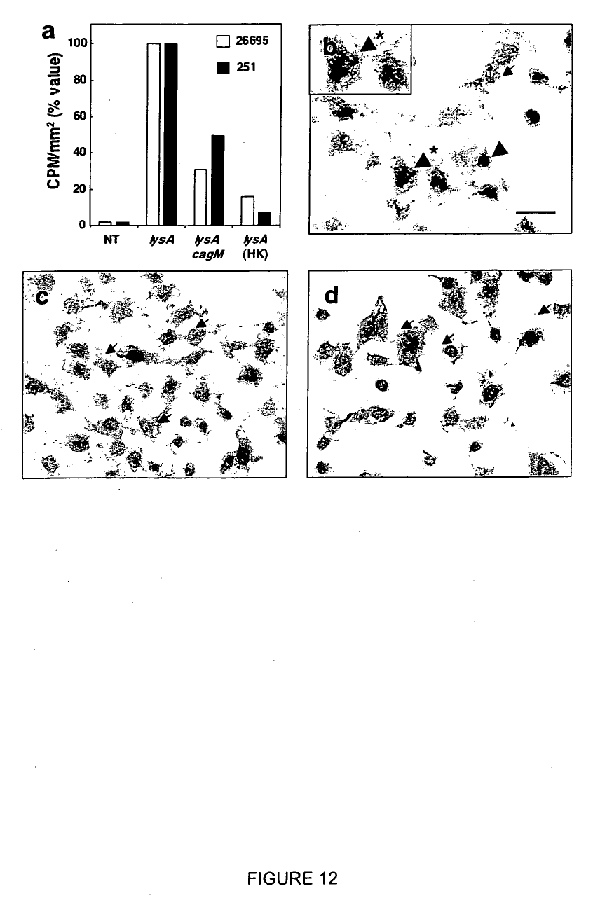

It was reasoned that H. pylori bacteria that are affected in PG turnover, and that hence release smaller quantities of PG muropeptides into the external medium, should be poor inducers of pro-inflammatory responses in epithelial cells. To investigate this, an H. pylori mutant deficient in lytic transglycosylase activity (sit, HP0645 (Tomb et al. (1997)), which is involved in bacterial muropeptide release, was generated and its effect on HEK293 cells studied. slt-deficient 26695 bacteria released up to 40% less disaccharide-tripeptide than the parental strain, yet liberated normal quantities of other major muropeptide forms (CC and IGB, unpublished data). Consistent with the postulated pro-inflammatory activity of H. pylori PG muropeptides, the H. pylori slt mutant induced significantly lower levels of NF-κB activation in HEK293 cells (FIG. 12a; P=0.004) and IL-8 synthesis in AGS cells (FIG. 12b; P=0.0009), when compared to the parental strain. H. pylori slt bacteria were shown, by CagA translocation and cell scattering assays in AGS cells, to have a functional type IV secretion system. Thus, the data demonstrated for the first time the effect of modifications in PG muropeptide release by replicating bacteria on Nod1-dependent responses in epithelial cells.

### EXAMPLE 15

### The H. pylori cagPAI Mediates Delivery of PG to Epithelial Cells

To address the mechanism by which H. pylori PG enters epithelial cells, cell co-culture experiments were performed with live H. pylori bacteria in which PG had been radio-labeled using ³H-mDAP. Since mDAP can be converted into either cellular proteins, via its conversion to l-lysine, or PG, the gene encoding diaminopimelate decarboxylase (LysA) activity, which is necessary for mDAP conversion into l-lysine, was inactivated. Enzymatic assays (n=3 experiments) of ³H-mDAP conversion into l-lysine in whole cell extracts revealed an activity of 0.57±0.07 nmol/min/mg for parental bacteria, as compared to no detectable activity for lysA mutant bacteria (unpublished data; Dr D. Blanot, Universitè de Paris-Sud, Orsay, France). This confirmed PG as the sole possible end-product for ³H-mDAP incorporation in H. pylori lysA bacteria.

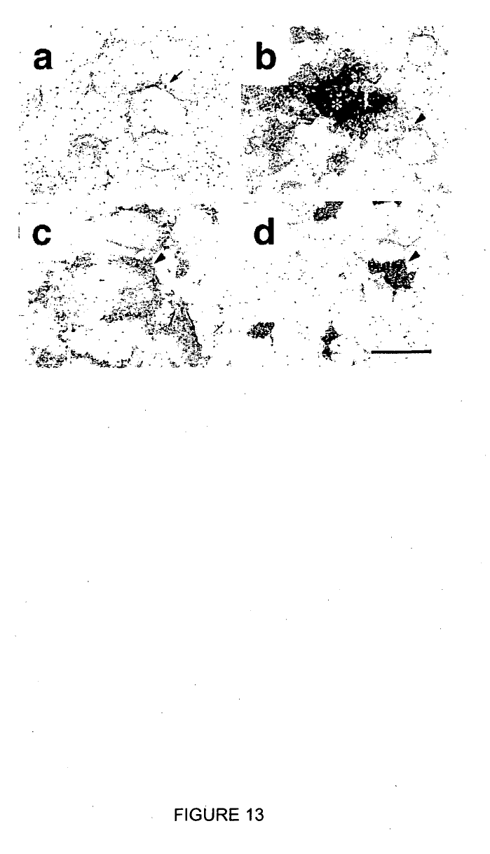

AGS co-culture experiments performed using ³H-labeled H. pylori bacteria revealed the highest levels of radioactivity in those cells that had been co-cultured with lysA-deficient H. pylori bacteria (FIG. 12a). Reduced radioactivity levels (50-90%) were recorded in cells that were incubated with bacteria in which the cag type IV secretion system had been rendered non-functional by gene disruption (lysA/cagM double mutants) or by heat inactivation (heat-killed lysA-deficient bacteria). The residual quantities of radioactivity present in cells co-cultured with lysA/cagM bacteria may be attributed to cagPAI-independent release of low levels of muropeptides (FIG. 12a). Nevertheless, large accumulations of radio-labeled PG particles were only observed in AGS cells that had been incubated with H. pylori lysA bacteria (FIG. 12a). This labeling appeared to be mainly perinuclear in nature and contrasted sharply with the more homogenous distribution seen for cells that had been co-cultured with bacteria lacking a functional cagPAI (FIG. 12c, d). As deletion of the cagPAI does not influence H. pylori adhesion to AGS cells (Odenbreit et al. (2002)), adherent bacteria are unlikely to contribute significantly to the observed differences in radioactive signal. Indeed, similar quantities of adherent H. pylori bacteria were detected by anti-H. pylori antibody labeling of cells following co-culture with either lysA or lysA/cagM bacteria (FIG. 13). In addition, heavy deposits of radio-labeled particles (FIG. 13b) did not appear to co-localize with antibody-reactive material present on the surfaces of the cells, thus further confirming the specificity of the ³H-labeling procedure for bacterial PG. These data therefore support the contention that a functional cagPAI is important for PG delivery to host epithelial cells, and are consistent with the putative role of internalized H. pylori PG in NF-κB responses in host cells.

### EXAMPLE 16

### Role of Nod1 in Host Defense Against cagPAI-Positive H. pylori Bacteria

The role of Nod1 in host defense against H. pylori infection in vivo was determined. To this end, Nod1^{-/-} mice and their wild-type (WT) counterparts were inoculated with H. pylori cagPAI-positive isolates. These were H. pylori 256, a clinical isolate (Philpott et al. (2002)), and H. pylori B128, a gerbil-adapted strain (Israel et al. (2001)). Both isolates colonize mice in moderate numbers (Philpott et al., (2002); Fox et al. (2003)) and induce NF-κB-dependent pro-inflammatory responses in AGS cells (Table 1).

TABLE 1
cagPAI genotypes and pro-inflammatory properties
of bacteria used in the study.

| | | | Induction of |
|---|---|---|---|
| | | | pro-inflammatory |
| | | cagPAI | responses in |
| Strains | | genotype | AGS cells¹ |
| H. pylori | 245 | +⁵ | +⁵ |
| | 251 | +⁵ | +⁵ |
| | 251cagA | cagA | NA² |
| | 251cagM | cagM | − |
| | 256 | +⁵ | +⁵ |
| | 26695 | +⁶ | +⁷ |
| | 26695slt | + | +³ |
| | 26695 lysA | + | + |
| | 26695lysAcagM | cagM | − |
| | 251 lysA | + | NA |
| | 251 lysAcagM | cagM | NA |
| | N6 | +⁸ | + |
| | SS1 | +⁹ | −⁵ |
| | B128 | +¹⁰ | + |
| | B128cagM | cagM | − |
| H. felis | CS1 (ATCC49179) | −⁴ | − |

¹The ability of bacterial strains to induce pro-inflammatory responses (NF-κB activation and/or IL-8 production) in AGS gastric epithelium cells was determined as described previously (Philpott et al. (2002)).
²NA, not available
³Data are presented in FIG. 11c, d
⁴H. felis cagPAI genotype was determined by low stringency Southern hybridization of H. felis chromosomal DNA using cagA- and cagF-specific genes.
⁵Philpott et al.(2002)
⁶Tomb et al. (1997)
⁷Fischer et al. (2001)
⁸Akopyants et al. (1998)
⁹Salama et al. (2000)
¹⁰Israel et al. (2001)

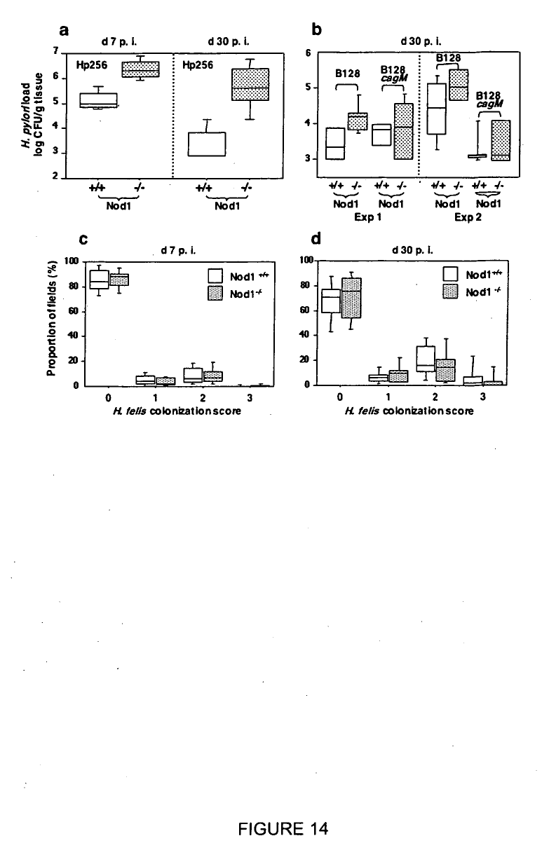

In addition, H. pylori B128 is naturally transformable. For comparative purposes, animals were also inoculated with a H. pylori B128cagM mutant and H. felis, a bacterium lacking a cagPAI homologue. H. felis colonizes mice in very high numbers and, unlike H. pylori strains, induces severe inflammatory changes in the early stages of murine infection (≦1 month) (Sakagami et al. (1996)). The colonization levels of H. pylori B128cagM and H. felis in Nod1^{-/-} mice did not differ significantly from those in WT animals (FIG. 14). In contrast, significant increases in the bacterial loads for H. pylori 256 were observed in Nod1^{-/-} mice at 7 and 30 days post-inoculation, when compared to WT animals (FIG. 14; P=0.008 and P=0.009, respectively). H. pylori B128 also colonized Nod1^{-/-} mice to a higher degree than WT animals, but the difference was less striking (FIG. 14). These data demonstrate a role for Nod1 in host defense against cagPAI-positive H. pylori isolates.

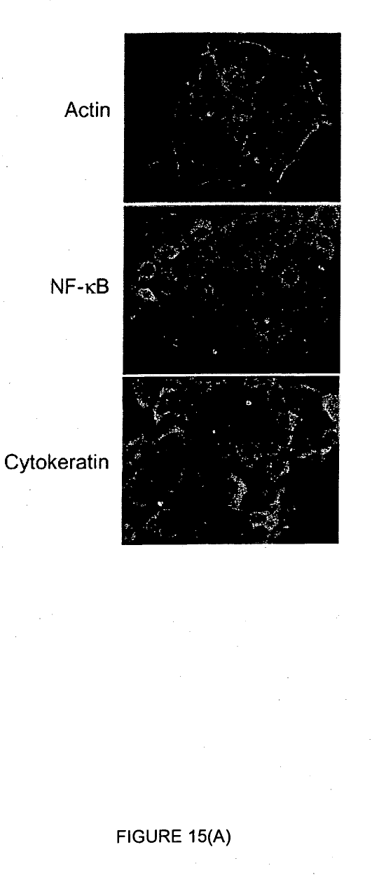

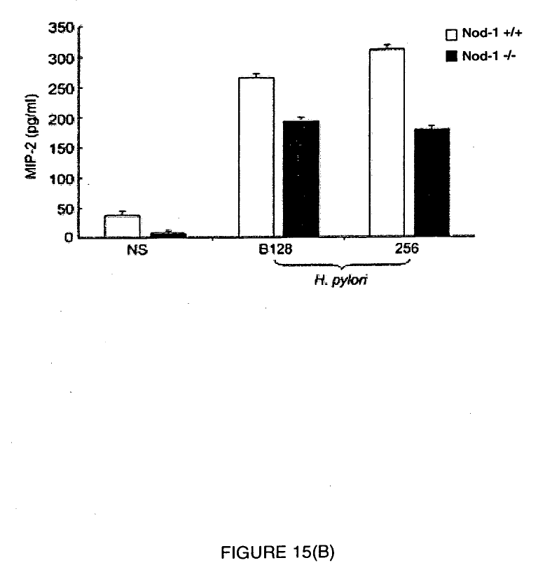

To address potential host immune defect(s) that may be responsible for the increased susceptibility of Nod1^{-/-} mice to H. pylori infection, we compared the ability of cultured gastric epithelial cells from Nod1^{-/-} and Nod1^{+/+} mice to mount pro-inflammatory responses against the bacterium (FIG. 15). For this, two types of read-out were measured in the cells: first, the nuclear translocation and binding of NF-κB, a key transcriptional regulator of genes involved in host immune functions; and second, the production of an NF-κB-dependent CXC chemokine, MIP-2, which has analogous biological activities in mice to human IL-8 (Remick et al. (2001); Widmer et al. (1993). H. pylori-stimulated cells did not appear to exhibit increased nuclear accumulation of the transcriptionally active form of NF-κB, containing p65 protein (FIG. 15A). Conversely, these cells synthesized high levels of MIP-2 in response to the bacterium. Furthermore, cultured gastric epithelial cells from Nod1^{-/-} mice produced significantly lower levels of MIP-2 in response to H. pylori stimulation, when compared to those from Nod1^{+/+} animals (P<0.0001; FIG. 15B). This suggests that Nod1^{-/-} mice may be affected in their ability to mount pro-inflammatory responses against this bacterial pathogen.

CXC chemokine assays. IL-8 and MIP-2 production by AGS and cultured primary epithelial cells, respectively, were determined from culture supernatants (24 h post-stimulation) using double sandwich ELISA kits (R&D Systems).

Statistical analysis. Data were analyzed using the Student's t-test and Mann-Whitney test, as appropriate. Differences in data values were considered significant for P≦0.05.

### REFERENCES

1. Akira, S., Takeda, K., Kaisho, T., Nat. Immunol. 2, 675 (2001).
2. Akopyants, N. S. et al. Analyses of the cag pathogenicity island of Helicobacter pylori. Mol Microbiol 28, 37-53 (1998).
3. Aras, R. A. et al. Plasticity of repetitive DNA sequences within a bacterial (Type IV) secretion system component. J Exp Med 198, 1349-60 (2003).
4. Athman, R., Niewöhner, J., Louvard, D. & Robine, S. Epithelial cells: Establishment of primary cultures and immortalization. in Methods in Microbiology, Vol. 31 (eds. Sansonetti, P. J. & Zychlinsky, A.) 96-113 (Academic Press Ltd, San Diego, 2002).
5. Backert, S. et al. Functional analysis of the cag pathogenicity island in Helicobacter pylori isolates from patients with gastritis, peptic ulcer, and gastric cancer. Infect Immun 72, 1043-56 (2004).
6. Backhed, F. & Hornef, M. Toll-like receptor 4-mediated signaling by epithelial surfaces: necessity or threat? Microbes Infect 5, 951-9 (2003).
7. Bäckhed, F. et al. Gastric mucosal recognition of Helicobacter pylori is independent of Toll-like receptor 4. J. Infect. Dis. 187, 829-836 (2003).
8. Bertin, J. et al. Human CARD4 protein is a novel CED-4/Apaf-1 cell death family member that activates NF-kappaB. J Biol Chem 274, 12955-8 (1999).
9. Blackburn, N. T., Clarke, A. J., J. Mol. Evol. 52, 78 (2001).
10. Blaser, M. J. et al. Infection with Helicobacter pylori strains possessing cagA is associated with an increased risk of developing adenocarcinoma of the stomach. Cancer Res 55, 2111-5 (1995).
11. Cario, E. et al. Commensal-associated molecular patterns induce selective toll-like receptor-trafficking from apical membrane to cytoplasmic compartments in polarized intestinal epithelium. Am J Pathol 160,165-73 (2002).
12. Cascales, E. & Christie, P. J. The versatile bacterial type IV secretion systems. Nat Rev Microbiol 1,137-49 (2003).
13. Censini, S. et al. cag, a pathogenicity island of Helicobacter pylori, encodes type I-specific and disease-associated virulence factors. Proc Natl Acad Sci USA 93, 14648-14653 (1996).
14. Chamaillard, M. et al. An essential role for NOD1 in host recognition of bacterial peptidoglycan containing diaminopimelic acid. Nat Immunol 4, 702-7 (2003).
15. Chevalier, C., Thiberge, J.-M., Ferrero, R. L. & Labigne, A. Essential role of Helicobacter pylori γ-glutamyltranspeptidase for the colonization of the gastric mucosa of mice. Molec. Microbiol. 31, 1359-1372 (1999).
16. Chin, A. I., et al., Nature 416, 190 (2002).
17. Choe, K. M., Werner, T., Stoven, S., Hultmark, D., Anderson, K. V., Science 296, 359 (2002).
18. Covacci, A. & Rappuoli, R. Helicobacter pylori: molecular evolution of a bacterial quasi-species. Curr Opin Microbiol 1, 96-102 (1998).
19. Coyle, A. J. et al. (submitted).
20. Crabtree, J. E., Ferrero, R. L. & Kusters, J. G. The mouse colonizing Helicobacter pylori strain SS1 may lack a functional cag pathogenicity island. Helicobacter 7, 139-40; discussion 140-1 (2002).
21. de Jonge, B. L., Chang, Y. S., Gage, D., Tomasz, A., J. Biol. Chem. 267, 11248 (1992).
22. Eckmann, L., Smith, J. R., Housley, M. P., Dwinell, M. B., Kagnoff, M. F., J. Biol. Chem. 275, 14084 (2000).
23. Evans, G. S., Flint, N., Somers, A. S., Eyden, B., Potten, C. S., J. Cell. Sci. 101, 219 (1992).
24. Ferrero, R. L. et al. The GroES homolog of Helicobacter pylori confers protective immunity against mucosal infection in mice. Proc. Natl. Acad. Sci. USA 92, 6499-6503 (1995).
25. Ferrero, R. L., Thiberge, J.-M., Huerre, M. & Labigne, A. Immune responses of specific-pathogen-free mice to chronic Helicobacter pylori (strain SS1) infection. Infect Immun 66, 1349-1355 (1998).
26. Fischer, W. et al. Systematic mutagenesis of the Helicobacter pylori cag pathogenicity island: essential genes for CagA translocation in host cells and induction of interleukin-8. Mol. Microbiol. 42, 1337-1348 (2001).
27. Fox, J. G. et al. Host and microbial constituents influence Helicobacter pylori-induced cancer in a murine model of hypergastrinemia. Gastroenterol. 124, 1879-90 (2003).
28. Garcia-Bustos, J. F., Dougherty, T. J., Antimicrob. Agents Chemother. 31, 178 (1987).
29. Gewirtz, A. T. et al. Helicobacter pylori flagellin evades toll-like receptor 5-mediated innate immunity. J Infect Dis 189, 1914-20 (2004).
30. Girardin, S. E. et al. CARD4/Nod1 mediates NF-kappaB and JNK activation by invasive Shigella flexneri. EMBO Rep 2, 736-42 (2001).
31. Girardin, S. E. et al. Nod1 detects a unique muropeptide from Gram-negative bacterial peptidoglycan. Science 300, 1584-1587 (2003).
32. Girardin, S. E. et al. Nod2 is a general sensor of peptidoglycan through muramyl dipeptide (MDP) detection. J Biol Chem 278, 8869-72 (2003).
33. Girardin, S. E., et al., J. Biol. Chem. 278, 8869 (2003).
34. Glauner, B. Separation and quantification of muropeptides with high-performance liquid chromatography. Anal Biochem 172, 451-64 (1988).
35. Gottar, M., et al., Nature 416, 640 (2002).
36. Hayashi, F., et al., Nature 410, 1099 (2001).
37. Hayashi, K. A., Anal. Biochem. 67, 503 (1975).
38. Heuermann, D. & Haas, R. A stable shuttle vector system for efficient genetic complementation of Helicobacter pylori strains by transformation and conjugation. MolGen Genet 257, 519-28 (1998).
39. Höltje. J.-H., Microbiol. Mol. Biol. Rev. 62, 181 (1988).
40. Hugot, J. P. et al., Nature 411, 599 (2001).
41. Inohara, N. et al. Nod1, an Apaf-1-like activator of caspase-9 and nuclear factor-kappaB. J Biol Chem 274, 14560-7 (1999).
42. Inohara, N. et al., J. Biol. Chem. 278, 5509 (2003).
43. Inohara, N., et al., J. Biol. Chem. 275, 27823 (2000).
44. Inohara, N., Ogura, Y., Chen, F. F., Muto, A., Nunez, G., J. Biol. Chem. 276, 2551 (2001).
45. Israel, D. A. et al. Helicobacter pylori strain-specific differences in genetic content, identified by microarray, influence host inflammatory responses. J Clin Invest 107, 611-20 (2001).
46. Jenks, P., C, C., C, E. & A, L. Identification of nonessential Helicobacter pylori genes using random mutagenesis and loop amplification. Res. Microbiol. 152, 725-734 (2001).
47. Josenhans, C., Eaton, K. A., Thevenot, T. & Suerbaum, S. Switching of flagellar motility in Helicobacter pylori by reversible length variation of a short homopolymeric sequence repeat in fliP, a gene encoding a basal body protein. Infect Immun 68, 4598-4603 (2000).
48. Kawai, T., Adachi, O., Ogawa, T., Takeda, K., Akira, S., Immunity 11, 115 (1999).
49. Kim, J. G., Lee, S. J. & Kagnoff, M. F. Nod1 is an essential signal transducer in intestinal epithelial cells infected with bacteria that avoid recognition by toll-like receptors. Infect Immun 72, 1487-95 (2004).
50. Kobayashi, K., et al., Nature 416, 194 (2002).
51. Laniece, P. et al. A new high resolution radioimager for the quantitative analysis of radiolabelled molecules in tissue section. J Neurosci Methods 86, 1-5 (1998).
52. Lee, H. K., Lee, J., Tobias, P. S., J. Immunol. 168, 4012 (2002).
53. Lee, S. K. et al. Helicobacter pylori flagellins have very low intrinsic activity to stimulate human gastric epithelial cells via TLR5. Microbes Infect 5, 1345-56 (2003).
54. Lemaitre, B., Nicolas, E., Michaut, L., Reichhart, J. M., Hoffmann, J. A., Cell 86, 973(1996) 10
55. Maeda, S. et al. Distinct mechanism of Helicobacter pylori-mediated NF-kB activation between gastric cancer cells and monocytic cells. J Biol Chem 276, 44856-44864 (2001).
56. McLaughlan, A. M., Foster, S. J., Microbiology. 144, 1359 (1998).
57. Medzhitov, R., Nat. Rev. Immunol. 1, 135 (2001).
58. Michel, T., Reichhart, J. M., Hoffmann, J. A., Royet, J., Nature 414, 756 (2001).
59. Moran, A. P., Helander, I. M. & Kosunen, T. U. Compositional analysis of Helicobacter pylori rough-form lipopolysaccharides. J. Bacteriol. 174, 1370-1377 (1992).
60. Odenbreit, S. et al. Translocation of Helicobacter pylori CagA into gastric epithelial cells by type IV secretion. Science 287, 1497-1500 (2000).
61. Odenbreit, S., Kavermann, H., Puls, J. & Haas, R. CagA tyrosine phosphorylation and interleukin-8 induction by Helicobacter pylori are independent from alpAB, HopZ and bab group outer membrane proteins. Int J Med Microbiol 292, 257-66 (2002).
62. Ogura, Y. et al., J. Biol. Chem. 276, 4812 (2001).
63. Ogura, Y. et al., Nature 411, 603 (2001).
64. Parsonnet, J. Helicobacter pylori: the size of the problem. Gut 43 Suppl 1, S6-9 (1998).
65. Pedron, T. et al. (submitted).
66. Peek, R. M., Jr et al. Heightened inflammatory response and cytokine expression in vivo to cagA+ Helicobacter pylori strains. Lab. Invest. 71, 760-770 (1995).
67. Philpott, D. J. et al. Reduced activation of inflammatory responses in host cells by mouse-adapted Helicobacter pylori isolates. Cell Microbiol 4, 285-96 (2002).
68. Philpott, D. J., Yamaoka, S., Israel, A. & Sansonetti, P. J. Invasive Shigella flexneri activates NF-kappa B through a lipopolysaccharide-dependent innate intracellular response and leads to IL-8 expression in epithelial cells. J Immunol 165, 903-14 (2000).
69. Philpott, D. J., Yamaoka, S., Israel, A., Sansonetti, P. J., J. Immunol. 165, 903 (2000).
70. Ramet, M., Manfruelli, P., Pearson, A., Mathey-Prevot, B., Ezekowitz, R. A., Nature 416, 644 (2002).
71. Remick, D. G. et al. CXC chemokine redundancy ensures local neutrophil recruitment during acute inflammation. Am. J. Pathol. 159, 1149-1157 (2001).
72. Rohde, M., Puls, J., Buhrdorf, R., Fischer, W. & Haas, R. A novel sheathed surface organelle of the Helicobacter pylori cag type IV secretion system. Mol Microbiol 49, 219-234 (2003).
73. Rougeot, C. et al. Targets for SMR1-pentapeptide suggest a link between the circulating peptide and mineral transport. Am J Physiol 273, R1309-20 (1997).
74. Sakagami, T. et al. Atrophic gastric changes in both Helicobacter felis and Helicobacter pylori infected mice are host dependent and separate from antral gastritis. Gut 39, 639-648 (1996).
75. Salama, N. et al. A whole-genome microarray reveals genetic diversity among Helicobacter pylori strains. Proc. Natl. Acad. Sc. USA 97, 14668-14673 (2000).
76. Sanchez Carballo, P. M., Rietschel, E. T., Kosma, P., Zahringer, U., Eur. J. Biochem. 261, 500 (1999).
77. Segal, E. D., Cha, J., Lo, J., Falkow, S. & Tompkins, L. S. Altered states: involvement of phosphorylated CagA in the induction of host cellular growth changes by Helicobacter pylori. Proc Natl Acad Sci USA 96, 14559-64 (1999).
78. Selbach, M. et al. The Helicobacter pylori CagA protein induces cortactin dephosphorylation and actin rearrangement by c-Src inactivation. Embo J 22, 515-28 (2003).
79. Selbach, M., Moese, S., Meyer, T. F. & Backert, S. Functional analysis of the Helicobacter pylori cag pathogenicity island reveals both VirD4-CagA-dependent and VirD4-CagA-independent mechanisms. Infect Immun 70, 665-71 (2002).
80. Skouloubris, S., Thiberge, J. M., Labigne, A. & De Reuse, H. The Helicobacter pylori Urel protein is not involved in urease activity but is essential for bacterial survival in vivo. Infect Immun 66, 4517-21 (1998).
81. Smith, M. F., Jr. et al. Toll-like receptor (TLR) 2 and TLR5, but not TLR4, are required for Helicobacter pylori-induced NF-kappa B activation and chemokine expression by epithelial cells. J Biol Chem 278, 32552-60 (2003).
82. Tomb, J. F. et al. The complete genome sequence of the gastric pathogen Helicobacter pylori. Nature 388, 539-547 (1997).
83. Widmer, U., Manogue, K. R., Cerami, A. & Sherry, B. Genomic cloning and promoter analysis of macrophage inflammatory protein (MIP)-2, MIP-1 alpha, and MIP-1 beta, members of the chemokine superfamily of proinflammatory cytokines. J. Immunol. 150, 4996-5012 (1993).
84. Xu, N., Huang, Z. H., de Jonge, B. L., Gage, D. A., Anal. Biochem. 248, 7 (1997).

What is claimed is:

1. A method for identifying a compound, which modulates the interaction between Nod1 and a Gram-negative bacteria comprising:
(a) contacting a Nod1 expressing cell with a cagPAI-positive H. pylori in the presence of a compound;
(b) contacting a Nod1 expressing cell with a cagPAI-positive H. pylori in the absence of said compound; and
(c) detecting the activation of a pro-inflammatory factor and/or production of a pro-inflammatory cytokine or chemokine in (a) and/or (b);
wherein altered activation of a pro-inflammatory factor and/or production of a pro-inflammatory cytokine or chemokine in (a) and/or (b) indicates that said compound modulates the interaction between Nod1 and the Gram-negative bacteria.

2. The method of claim 1, wherein said pro-inflammatory factor is NF-κB and said cytokine or chemokine is an NF-κB-dependent cytokine or chemokine.

3. The method of claim 2, wherein said NF-κB-dependent cytokine or chemokine is IL-8 or MIP-2.

4. The method of claim 1, wherein said Nod1 expressing cell is an epithelial cell.

5. The method of claim 1, wherein the activation of a pro-inflammatory factor and/or production of a pro-inflammatory cytokine or chemokine in (a) is increased.

6. The method of claim 1, wherein the activation of a pro-inflammatory factor and/or production of a pro-inflammatory cytokine or chemokine in (a) is decreased.

7. The method of claim 1, wherein said detecting the activation of a pro-inflammatory factor and/or production of a pro-inflammatory cytokine or chemokine in (a) and/or (b) comprises detecting NF-κB activation.

8. The method of claim 7, wherein said NF-κB activation is detected by a bioluminescent signal.

9. The method of claim 1, wherein said activation of a pro-inflammatory factor and/or production of a pro-inflammatory cytokine or chemokine in (a) and/or (b) is abrogated by treating said Nod1 expressing cell with siRNA against Nod1.

10. The method of claim 9, where said siRNA against Nod1 comprises the polynucleotide 5′-ACAACTTGCTGAAGAATGACT-3′ [SEQ ID NO: 1].

11. The method of claim 1, wherein said activation of a pro-inflammatory factor and/or production of a pro-inflammatory cytokine or chemokine in (a) and/or (b) is abrogated by treating said Nod1 expressing cell with dominant-negative Nod1.

12. A method for identifying a compound, which modulates the interaction between Nod1 and a Gram-negative bacteria comprising:
(a) contacting a Nod1 expressing cell with a cagPAI-positive H. pylori in the presence of a compound;
(b) contacting a Nod1 expressing cell with a cagPAI-positive H. pylori in the absence of said compound;
(c) contacting a Nod1 expressing cell with a cagPAI-negative H. pylori in the presence of said compound;
(d) contacting a Nod1 expressing cell with a cagPAI-negative H. pylori in the absence of said compound; and
(e) detecting the activation of a pro-inflammatory factor and/or production of a pro-inflammatory cytokine or chemokine in (a), (b), (c), and (d);
wherein altered activation of a pro-inflammatory factor and/or production of a pro-inflammatory cytokine or chemokine (a) and/or (b) and/or (c) and/or (d) indicates that said compound modulates the interaction between Nod1 and the Gram-negative bacteria.

13. The method of claim 12, wherein said pro-inflammatory factor is NF-κB and said cytokine or chemokine is an NF-κB dependent cytokine or chemokine.

14. The method of claim 12, wherein said NF-κB dependent cytokine or chemokine is IL-8 or MIP-2.

15. A method for detecting a dysfunction of the inflammatory and/or apoptosis pathway in which Nod1 is involved, comprising:
(a) bringing a cagPAI-positive H. pylori into contact with a cell in which the dysfunction of the inflammatory and/or apoptosis pathway in which Nod1 is involved, is suspected;
(b) bringing a cagPAI-negative H. pylori into contact with a cell in which the dysfunction of the inflammatory and/or apoptosis pathway in which Nod1 is involved, is suspected, and
(c) evaluating activation of a pro-inflammatory factor and/or production of a pro-inflammatory cytokine or chemokine in (a) and (b),
wherein similar levels of activation of a pro-inflammatory factor and/or production of a pro-inflammatory cytokine or chemokine in (a) and (b) indicates dysfunction of a molecule of the inflammatory and/or apoptosis pathway in which Nod1 is involved.

16. The method of claim 15, wherein said pro-inflammatory factor is NF-κB and said cytokine or chemokine is an NF-κB dependent cytokine or chemokine.

17. The method of claim 16, wherein said NF-κB dependent cytokine or chemokine is IL-8 or MIP-2.

18. The method of claim 15, wherein said cell is an epithelial cell.

19. The method of claim 15, wherein said evaluating activation of a pro-inflammatory factor and/or production of a pro-inflammatory cytokine or chemokine in (a) and (b) comprises detecting NF-κB activation.

20. The method of claim 19, wherein NF-κB activation is detected by a bioluminescent signal.

21. A method for inactivating Nod1 in a Nod1 expressing cell comprising administration of siRNA against Nod1 in an amount sufficient to cause inactivation of Nod1.

22. The method of claim 21, wherein said siRNA against Nod1 comprises the polynucleotide sequence 5′-ACAACTTGCTGAAGAATGACT-3′ [SEQ ID NO: 1].

23. The method of claim 21, wherein said Nod1 expressing cell is an epithelial cell.

24. The method of claim 21, wherein said Nod1 expressing cell is transfected with about 50-500 ng of a construct comprising a sequence specific for CARD in human nod1.

25. The method of claim 24, wherein said construct comprises the polynucleotide sequence 5′-ACAACTTGCTGAAGAATGACT-3′ [SEQ ID NO: 1].

26. The method of claim 25, wherein said Nod1 expressing cell is an epithelial cell.

27. A method for assaying whether a Gram-negative bacteria is cagPAI-positive comprising the steps of:
(a) contacting a Gram negative bacteria with a cell line expressing Nod1;
(b) contacting a Gram negative bacteria with a cell line not expressing Nod1; and
(c) evaluating activation of a pro-inflammatory factor and/or production of a pro-inflammatory cytokine or chemokine in (a) and (b);
wherein altered activation of a pro-inflammatory factor and/or production of a pro-inflammatory cytokine or chemokine in (a) and/or (b) indicates that said Gram-negative bacteria is cagPAI-positive.

28. The method of claim 27, wherein said pro-inflammatory factor is NF-κB and said cytokine or chemokine is an NF-κB dependent cytokine or chemokine.

29. The method of claim 28, wherein said NF-κB dependent cytokine or chemokine is IL-8 or MIP-2.

30. The method of claim 27, wherein said evaluating activation of a pro-inflammatory factor and/or production of a pro-inflammatory cytokine or chemokine in (a) and (b) comprises detecting NF-κB activation.

31. The method of claim 30, wherein said NF-κB activation is detected by a bioluminescent signal.

32. The method of claim 27, wherein said altered activation of a pro-inflammatory factor and/or production of a pro-inflammatory cytokine or chemokine in (a) and/or (b) is abrogated by treatment of said cell line expressing Nod1 with siRNA against Nod1.

33. The method of claim 32, wherein said siRNA against Nod1 comprises the polynucleotide 5′-ACAACTTGCTGAAGAATGACT-3′ [SEQ ID NO: 1].

34. The method of claim 27, wherein said altered activation of a pro-inflammatory factor and/or production of a pro-inflammatory cytokine or chemokine in (a) and/or (b) is abrogated by treatment of said cell line expressing Nod1 with dominant-negative Nod1.

35. A method of inducing a pro-inflammatory response and/or apoptosis in a cell containing intracellular Nod1, wherein the method comprises contacting said cell with H. pylori cell-free PG, a fragment thereof, or a related molecule thereof.

36. The method of claim 35, wherein said cell is a mammalian cell.

37. The method of claim 36, wherein said mammalian cell is a gastric epithelial cell.

38. The method of claim 35, wherein the H. pylori PG fragment is H. pylori MTP or a molecule related to H. pylori MTP.

39. The method of claim 35 comprising activating an NF-κB signaling pathway in said cells.

40. A composition that comprises a biologically acceptable carrier and a biologically effective amount of H. pylori PG, H. pylori MTP, or a molecule related to H. pylori MTP.

41. A method for preventing or treating abnormal level or rate of apoptotic cell death or inflammation, comprising administering the composition of claim 40 in a therapeutically effective amount to a human or animal in need thereof.

42. A method for preventing or treating a Gram-negative bacteria infection, comprising administering the composition of claim 40 in an effective amount to a human or animal in need thereof.


SEQUENCE LISTING (7 sequences)

SEQ ID NO: 1 - DNA, 21 bp, Artificial Sequence
  Description of Artificial Sequence Synthetic oligonucleotide
acaacttgctgaagaatgact

SEQ ID NO: 2 - DNA, 21 bp, Artificial Sequence
  Description of Artificial Sequence Synthetic oligonucleotide
gcaagctgaccctgaagttca

SEQ ID NO: 3 - DNA, 18 bp, Artificial Sequence
  Description of Artificial Sequence Synthetic primer
cctgacaaggtccgcaaa

SEQ ID NO: 4 - DNA, 20 bp, Artificial Sequence
  Description of Artificial Sequence Synthetic primer
gtccatgtagatctcctcca

SEQ ID NO: 5 - DNA, 20 bp, Artificial Sequence
  Description of Artificial Sequence Synthetic primer
gggtcagaaggattcctatg

SEQ ID NO: 6 - DNA, 20 bp, Artificial Sequence
  Description of Artificial Sequence Synthetic primer
ggtctcaaacatgatctggg

SEQ ID NO: 7 - protein, 5 aa, Unknown Organism
  Description of Unknown Organism Bacterial peptide motif
LPXTG